(19) United States
(12) Patent Application Publication
(10) Pub. No.: US 2008/0259519 A1
(43) Pub. Date: Oct. 23, 2008

(54) Highly-Aqueous, Non-Respirable Aerosols Containing Biologically-Active Ingredients, Method of Making, and Device Therefor

(75) Inventors: Ada S. Cowan, Lewis Center, OH (US); Sreedhara Alavattam, Fremont, CA (US); Beverly A. Piatt, Columbus, OH (US); James E. Dvorsky, Norwich Township, OH (US); William M. Fountain, Circleville, OH (US); Mark R. Wilson, Columbus, OH (US)

Correspondence Address: BATTELLE MEMORIAL INSTITUTE, 505 KING AVENUE, COLUMBUS, OH, 43201-2693, US

(73) Assignee: Battelle Memorial Institute, Columbus, OH (US)

(21) Appl. No.: 11/662,833

(22) PCT Filed: Sep. 14, 2005
(86) PCT No.: PCT/US05/33108

§ 371 Date: Feb. 7, 2008

Related U.S. Application Data
(60) Provisional application No. 60/609,791, filed on Sep. 14, 2004.

(30) Foreign Application Priority Data
Jul. 7, 2005 (US) 10/541681

(51) Int. Cl. B05B 5/053; A01N 25/06; A01P 1/00; A01P 3/00; A01P 13/00; A01P 15/00; A01P 21/00

(52) U.S. Cl. 361/228; 514/772; 504/362

(57) ABSTRACT

A non-respirable aerosol, particularly a non-respirable aerosol comprising a biologically-effective amount of a biologically-active agent dissolved, emulsified, or suspended in a highly-aqueous liquid carrier vehicle. The highly-aqueous liquid carrier vehicle comprises about 60 wt % to about 100 wt % water, about 0 wt % to about 40 wt % of a co-solvent, about 0.05 wt % to about 10 wt % of an acceptable surfactant, and about 0 wt % to about 10 wt % of an excipient. The non-respirable aerosol is substantially monodisperse when dispensed from a sprayhead assembly comprising a preferably linear array of a plurality of nozzles and at least one counter electrode adapted to substantially equalize the charge fields of the plurality of nozzles.

### CROSS-REFERENCE TO RELATED APPLICATION(S)

This Application claims priority to i) Provisional U.S. Pat. App. No. 60/609,791, filed Sep. 14, 2004, and ii) Int'l App. No. PCT/US2004/000556, U.S. patent application Ser. No. 10/541,681, now U.S. Pat. No. ______, which claims priority to Provisional U.S. Pat. App. Nos. i) 60/439,254, filed Jan. 10, 2003, now abandoned, ii) 60/439,257, filed Jan. 10, 2003, now abandoned, and iii) 60/439,606, filed Jan. 11, 2003, now abandoned, the contents of which are incorporated herein by reference as if fully rewritten herein.

### STATEMENT REGARDING FEDERALLY-SPONSORED RESEARCH OR DEVELOPMENT

Not applicable.

### INCORPORATION-BY-REFERENCE OF MATERIAL SUBMITTED ON A CD

### Not applicable.

### BACKGROUND OF THE INVENTION

1. Field of the Invention

The present invention relates to the field of non-respirable aerosols, particularly non-respirable aerosols produced from very low-resistivity liquid compositions, particularly very low-resistivity aqueous liquid compositions, using electrohydrodynamic (EHD) means, including an improved nozzle, for generating small, uniform, non-respirable aerosol particles comprising biologically-active agents, as well as to methods of using such liquid formulations to deliver biologically-active agents to a target surface.

2. Description of Related Art

Creating non-respirable particles of highly-aqueous liquid formulations using EHD presents some unique problems. To effect the Taylor cone, which is the hallmark of EHD, the liquid must be subjected to a charge sufficient to overcome the surface tension of the liquid and the liquid breaks apart (a process called comminution). In a highly-aqueous formulation, the surface tension which must be overcome is generally very high. In addition, the resistivity of the formulation is very low which inhibits formation of a charge on the liquid and the formation of the Taylor cone. To overcome the surface tension in a highly-aqueous, high-resistivity formulation, a high charge is required. This, in turn, creates particles of very small size, at very low delivery rates, and generally non-uniform dispersions. Thus, there is a need to create larger, non-respirable particles of highly-aqueous, high-resistivity formulations that can be utilized in situations where respiration would be harmful.

Handheld electrohydrodynamic aerosolization/spraying means are known in the art. U.S. Pat. No. 6,397,838 to Zimlich et al. describes a handheld, EHD pulmonary aerosol delivery device that produces a cloud of aerosolized liquid particles having a mono-disperse, respirable particle size and mean zero velocity. As described in Zimlich, the aerosol particles are such that at least about 80 percent have a diameter of less than or equal to about 5 microns.

U.S. Pat. No. 4,381,533 to Coffee describes an EHD spray device, principally for use in crop spraying. A stated essential component of the Coffee spray device is a circular field intensifying electrode, sited annularly adjacent to the circular sprayhead. In use, it is stated to reduce the incidence of corona discharge which interferes with spray production and thereby allows lower electric field strengths to be used during spray generation.

U.S. Pat. No. 6,503,481 to Thurston et al. teaches a method for delivering a biologically-active material to the respiratory tract of a patient in need of treatment comprising the steps of producing a respirable aerosol of a liquid composition using an EHD spraying means and administering the aerosol to the pulmonary tract of a patient via inhalation of the aerosol. The aerosol comprises a pharmaceutically-effective amount of an active agent in a carrier liquid in which the active agent is dissolved, emulsified, or suspended. Specific liquid medicament formulations are described which are useful in the methods of the invention.

Various liquid medicament formulations suitable for aerosolization using an EHD device and administration to a patient by pulmonary delivery are described in the following U.S. Pat. Pub. Nos. 2002/0102218 to Cowan and 2002/0110524 and 2003/0185762 to Cowan et al. None of these publications disclose the particular non-respirable aerosols described and claimed herein.

Finally, especially with multiple-nozzle arrays, and particularly with multiple-nozzle arrays that are substantially linear, the field intensity at each nozzle, or spray site, varies from site to site due to the effect each nozzle has on adjacent nozzles. Such uneven field intensity results in uneven aerosolization and uneven particle size, which, in turn, results in a much greater particle size distribution. This interferes with the spraying of aqueous formulations using multiple nozzle arrays, and particle size variability can also be undesirable when spraying biologically-active materials.

There is, therefore, a need for improved highly-aqueous non-respirable aerosols, methods of making same, and devices therefor.

### BRIEF SUMMARY OF THE INVENTION

The invention is directed to non-respirable aerosols useful for delivery of a biologically-active agent to a target surface, as well as to methods of treating a target surface using such biologically-active aerosols, and further to highly-aqueous liquid carrier vehicles for biologically-active agents which are suitable for aerosolization using an EHD spraying means. An improved sprayhead assembly for generating such aerosols is also disclosed.

One object of the present invention is to provide an aqueous liquid carrier vehicle for direct delivery of an aerosol having a particle size of between about 60 μm and about 800 μm and containing a dissolved or suspended optionally biologically-active agent, comprising about 60 weight percent to about 100 weight percent water, about zero weight percent to about 40 weight percent co-solvent, about 0.05 weight percent to about 10 weight percent of an acceptable surfactant, and about zero weight percent to about 10 weight percent of an optionally biologically-acceptable excipient, wherein the liquid carrier vehicle has a resistivity of about 0.05 ohm-m to about 20 ohm-m, a surface tension of about 20 dynes/cm to about 100 dynes/cm, and a viscosity of about 0.1 cPs to about 100 cPs.

It is a further object of the present invention to provide an aqueous liquid carrier vehicle for direct delivery of an aerosol having particles having a GSD of about 1.10 to about 1.65.

It is a further object of the present invention to provide an aqueous liquid carrier vehicle comprising a surfactant having a surface tension of about 30 dynes/cm or less.

Another object of the present invention is to provide an aerosol, using EHD means, having a particle size between about 60 μm and about 800 μm and comprising a biologically-effective amount of a biologically-active agent dissolved, suspended, or emulsified in the aqueous liquid carrier vehicle described herein above.

Yet another object of the present invention is to provide a method for delivering a biologically-active agent to a target surface in need of treatment, comprising the steps of preparing an aqueous liquid carrier vehicle as described herein above, dissolving, suspending, or emulsifying a biologically-effective amount of the biologically-active agent in the liquid carrier vehicle, producing an aerosol of the solution or suspension using EHD means, wherein the aerosol particle size in about 60 μm to about 800 μm and applying the aerosol to the target surface.

Yet another object of the present invention is to provide a sprayhead assembly for EHD spraying, comprising a nozzle array, the nozzle array comprising a plurality of nozzles, preferably configured in a substantially linear arrangement, and comprising at least one, preferably a plurality or array, of inner nozzles and preferably at least one first and at least one second nozzle (also referred to herein as a first array and a second array of outer nozzles, respectively), at least one field-equalizing counter electrode, the counter electrode in charge communication with the nozzle array and comprising a first end portion, a second end portion, and a central portion therebetween, wherein the central portion is positioned closer to the array of inner nozzles than the first end portion is positioned to the first array of outer nozzles and than the second end portion is positioned to the second array of outer nozzles.

It is yet another object of the present invention to provide a method of substantially equalizing the fields about a plurality of nozzles, the method comprising the steps of providing a plurality of nozzles, charging at least two of the plurality of nozzles, wherein the charge on the at least two nozzles in unequal, providing at least one counter electrode, and applying a charge or ground to the at least one counter electrode, whereby the field on the at least two nozzles becomes substantially equal.

In EHD practice, as described hereinabove, the liquid is subject to a charge which causes it to form a Taylor cone and, subsequently, to comminute. It is the charge on the liquid at the Taylor cone that, in part, controls the comminution. An electrical charger is placed in electrical communication with the liquid, either directly via the fluid itself, or indirectly via the nozzle. It is well within the ability of EHD spraying to provide a substantially monodisperse aerosol when using one spray site. In practice, however, a higher flowrate is often desired than is practically achievable with a single site. In a multi-site nozzle array, especially one that is substantially linear, the charge experienced by the liquid in the Taylor cone may be affected by the charge on nearby sites. Thus, not all sites (Taylor cones) will produce the same aerosol distribution.

The present invention introduces a counter electrode design that can alter the charge at each site by affecting the field at the spray site in the vicinity of the Taylor cone in a manner which alters, influences, and can make more equal or substantially equal, the charge at each spray site, cause the collective aerosol to be more monodisperse, and provide control of droplet size. This ability of the counter electrode to effect the charge at the spray sites without being in electrical contact with the spray site is referred to herein as “charge communication”.

It may be understood that not only may varying the shape and charge of the counter electrode one may effect charge communication with a series of spray sites, but also that such charge communication can be adjusted on a given spray head mechanically or electrically. Mechanical adjustment is possible by placing the counter electrode of the present invention (whether a curved filament or a series of individual counter electrodes) on adjustable or movable supports which permit adjustment in the three dimensions of height relative to the spray sites, distance of the electrode to the spray sites, or curvature of the counter electrode. Where a series of individual electrodes comprise the counter electrode in accordance with the present invention, each of the individual electrodes of the counter electrode may be separately adjustable.

As well, electrical adjustment of the counter electrode of the present invention alone or in combination with mechanical adjustment features, permits controlled application of charge communication to tune an apparatus as needed for spraying a particular material, and to create desired dispersion characteristics. To this end, the voltage on the counter electrode can be changed as desired. Alternatively, the charge on ones of a series of counter electrodes arranged in accordance with the present invention can be separately controlled and thus dynamically adjusted to vary the charge communication and influence spray site performance. Electronic controllers may be useful in this regard to provide dynamic adjustment during use.

### BRIEF DESCRIPTION OF THE SEVERAL VIEWS OF THE DRAWINGS

The following detailed description of the embodiments of the invention will be more readily understood when taken in conjunction with the following drawing, wherein:

FIGS. 1-9 are plots of particle size data for the various examples described herein.

FIGS. 10-12 illustrate a nozzle assembly according to an aspect of the present invention.

FIGS. 13-21 illustrate various embodiments of a nozzle assembly according to another aspect of the present invention.

FIGS. 22-51 picture FEA results of various embodiments of a nozzle assembly according to another aspect of the present invention.

### DETAILED DESCRIPTION OF THE INVENTION

In one embodiment of the present invention, an aqueous

As used herein, the term “electrohydrodynamic” or “EHD” may also be referred to as “electric field effect technology” or “EFET” and these terms are used interchangeably. Dispensing devices are known which produce a finely divided spray of liquid particles EFET (EHD) means. These sprayers have found use in many areas, including, without limitation, medicine for the administration of medicaments and biologicals by topical application or inhalation, in agriculture for crop spraying, in consumer markets for consumer products, and in industry for spraying coatings, paints, and other materials used in manufacturing processes.

The EHD spraying means described herein may be stationary or handheld. Such devices are “stationary” in the respect that their size prevents them from being easily held and carried by the user. Stationary EHD devices may be portable if moved on a cart, dolly, or vehicle such as a truck or an airplane. In many of the applications described herein, it is advantageous that the EHD device be small, portable, and handheld. As an example, an EHD device about the size of a cell phone would enable the user/applicator to use apply the biologically active aerosols in a variety of locations where it would be inconvenient to move a larger device. For example, a portable, handheld EHD device is ideal for treating one's clothing in a wooded or field environment where there may be deer ticks infected with the bacterium Borrelia burgdorferi, which causes Lyme Disease and which is transmitted to humans by the bite of an infected deer tick.

In a typical EHD device, a fluid delivery means delivers fluid to be aerosolized to a nozzle and the fluid or the nozzle is maintained at high electric potential. One type of nozzle used in EHD devices is a capillary tube capable of conducting electricity. An electric potential is placed on the capillary tube which charges the fluid contents or upon the fluid itself such that, as the fluid emerges from the tip or end of the capillary tube, a so-called Taylor cone is formed. This cone shape results from a balance of the forces of electric charge on the fluid and the surface tension of the fluid Desirably, the charge on the fluid overcomes the surface tension and at the tip of the Taylor cone, a thin jet of fluid forms and subsequently and rapidly separates a short distance beyond the tip into an aerosol. Studies have shown that the aerosol from a single capillary nozzle can have a substantially uniform particle size and a high velocity leaving the tip but that it quickly decelerates to a very low velocity a short distance beyond the tip.

EHD sprayers produce charged particles at the tip of the nozzle. Depending upon the use, these charged particles can be partially- or fully-neutralized (with, for example, a discharge electrode in the sprayer device). When the EFET device is used to deliver therapeutic, respirable aerosols, it is preferred that the aerosol be completely electrically neutralized prior to inhalation by the user to permit the aerosol to reach the pulmonary areas where the particular therapeutic formulation is most effective. In the case of a non-therapeutic, non-respirable aerosol such as the subject of the present invention, typically the aerosol is intended to be deposited on a target surface, and an EFET sprayer without means for discharging or with means for only partially discharging an aerosol might be preferred since the aerosol would have a residual electric charge as it leaves the sprayer so that the particles would be attracted to and tightly adhere to the target surface.

The nozzle assembly of an EHD spray device may include one or more, preferably two for a linear nozzle array, so-called “dummy” electrodes. In practice, a dummy electrode is placed at each end of the nozzle array. While the dummy electrodes are charged similarly to the active spray sites, no fluid is supplied to them. They serve only to help balance the electric charges, especially at the outermost spray sites in a linear array. See, e.g., U.S. Pat. No. 6,302,331 to Dvorsky et aL. cited above.

Int'l App. No. PCT/US2004/000556, to which the instant application claims priority, the contents of which are included herein by reference as if fully rewritten herein, discloses a spray-shaping mechanism comprising parallel counter electrodes. These counter electrodes may be employed in “localizing” the electric field that is produced at the spray site. The counter electrodes may effectively boost the velocity of the EHD spray forward, as well as shape or split the spray toward a desired target. This feature is capable of presenting a more uniform field to each spray site. Alternatively, a counter electrode may be referred to as a “reference” electrode. What is intended, however, is that the electrode has a potential relative to the spray site. As will be appreciated by those skilled in the art, no implication is intended as to any specific polarity or relativity to earth ground or other external reference for the counter electrode. The key is that the counter electrode has a potential relative to the spray site. As described in more detail herein below, counter electrodes have been developed which are particularly suitable to effecting a more uniform field about each spray site in a substantially linear nozzle array.

Various EHD devices are known in the art, for example, U.S. Pat. Nos. 6,302,331 to Dvorsky et al., 6,105,877, 6,457,470, 6,386,195, and 6,252,129 to Coffee, and 6,595,208 to Coffee et al. Although, the various patents disclose different methods for obtaining aerosols having an aerosol particle size of in the range of from 0.1 um to 50 um, very little direction is provided regarding suitable carrier liquids or improving spray site field uniformity.

The term “aqueous liquid carrier vehicles” as used herein refers to the liquid carrier vehicle in which the biologically-active agent to be applied to a target surface is dissolved or suspended. The aqueous liquid carrier vehicle is required to contain at least about 60 weight percent to about 100 weight percent water, preferably from about 85 weight percent to about 100 weight percent water, and more preferably from about 90 weight percent to about 100 weight percent water. The term “highly aqueous” is used herein to describe aqueous liquid carrier vehicles of the invention containing from about 90 weight percent to about 99 weight percent water and more preferably from about 95 weight percent to about 100 weight percent water.

The aerosols of the invention can be used to deliver a “biologically-active agent” to a target surface. The term “target surface” as used herein may be any surface that benefits from treatment of a biologically-active agent with a soft cloud of a non-respirable aerosol according to the invention. As used herein, the term “target surface” does not refer to an interior tissue surface in a human or animal body such as the lungs or oral, vaginal, or rectal cavities. The target surface may be for example, plants, the soil (ground) around plants, the leaves and stems of plants, the eyes, skin, coat, hide, or hide of animals such as cats, dogs, and horses, the skin, eyes, and hair of humans, the clothing of humans, and hard surfaces such as walls, floors, tables, desks, beds, and other furnishings, manufacturing and building infrastructure, and the like found in hospitals, nursing homes, schools, and restaurants.

As used herein, the term “biologically-active agent” refers to an agent or combination of agents that may be used in agriculture, horticulture, veterinary medicine, personal animal, or human care, disinfecting, and other applications where it is desirable to deliver a biologically-active agent to a target surface. The biologically-active agents contemplated for use in the aerosols and methods of the invention include but are not limited to herbicides, plant growth regulators, insecticides, fungicides, miticides, biocides, antibacterials, antivirals, anti-inflammatories, disinfectants, ocular decongestants, skin and eye treatments, and the like.

Illustrative, but non-limiting examples of the aerosols prepared as described herein are aerosols useful to deliver insecticides and fungicides to trees and shrubs, plants such as roses, orchids, violets, and other valuable flowering plants, as well as to deliver herbicides to bed plantings and home gardens, especially when handheld, battery powered, portable EHD devices are used to produce the aerosol. The aerosols and methods of the invention can be used to apply anti-tick, flea, and mite active agents to the coat of mammals such as dogs, cats, and horses, the skin and hair of humans, and the outer clothing of humans to protect against fleas, ticks, and mites. The aerosols of the invention can be used to apply disinfectant agents to hard surfaces in schools, restaurants, hospitals, businesses, stores, manufacturing facilities, and the home. In schools, for example, the aerosols may be use to treat desks and cafeteria tables to prevent the spread of viruses and bacterial, especially in influenza season.

Illustrative, but non-limiting examples of specific biologically-active agents useful in the aerosols and methods of the invention include: herbicides e.g., (2,4,5-trichlorophenoxy)acetic acid, (4-chloro-2-methylphenoxy)acetic acid, (2,4-dichlorophenoxy)acetic acid, 4-(4-chloro-o-tolyloxy)butyric acid, fluazifop-p-butyl (Ornamec®, Gordon Corp, Kansas City, Mo.), pelargonic acid (Scythe®, Mycogen Corp., San Diego, Calif.), and isopropylamine salt of N-(phosphonomethyl)glycine (Roundup®, Scotts, Marysville, Ohio or Glyphomax®, Dow Agrosciences, Indianapolis, Ind. ); fungicides e.g., manganese ethylene bisdithiocarbamate (Maneb), 1-(4-chlorophenoxy)-3,3-dimethyl-1-(1H-1,2,4-triazol-1-yl)-2-butanone (Strike®, Olympic Horticultural Products, Mainland, Pa.), azoxystrobin (Amistar®, Syngenta, Basel, CH), andtrifloxystrobin (Compass™, Bayer CropScience, Research Triangle Park, N.C.); insecticides, e.g., Bacillus thuringiensis (B.t.) (sold under the trade names Dipel® (Valent Corp, Dublin, Calif.), Thuricide® (Bonide Products, Oriskany, N.Y.), Bactospeine® (PBI/Gordon, Kansas City, Mo.), Leptox, Novabac, and Bug Time); synthetic pyrethroids, e.g., permethrin, cypermethrin, fenvalerate/esfenvalerate, tralomethrin, bifenthrin, cyfluthrin, and lambda-cyhalothrin, O,O-Diethyl 0-(2-isopropyl-6-methyl-4-pyrimidinyl) phosphorothioate (diazinon); treatments for fleas, ticks, and lice, e.g., lindane and malathion (headlice, pubic lice), permethrin (ticks), N,N-diethyl-meta-toluamide (DEET) (mosquitoes), fenthion, and cythioate (fleas); and disinfectants, e.g., 3,4′,5 tribromosalicylanilide (tribromsalan).

The biologically-active agents described herein are present in the aerosols of the invention at a “biologically-effective amount”. As would be recognized by one skilled in the art, by “biologically-effective amount” is meant an amount of a biologically-active agent that is sufficient to provide the result sought. In general, from about 0.01 weight percent to about 50 weight percent of the biologically-active agent will be present in the liquid carrier vehicle. Specific details of the effective dosage or concentration of a particular active agent may be found in its product labeling, e.g., the package insert if the active agent is regulated by the United States Food and Drug Administration (FDA) (see, 21 CFR § 201.56 & 201.57) or the labeling approved by the United States Environmental Protection Agency (EPA) if the active agent is, e.g., a herbicide, insecticide, miticide, and the like, which is covered by the rules and regulations of the EPA.

When a biologically-active agent is added to the aqueous liquid carrier vehicle, a solution is produced if the active agent is soluble in the liquid carrier vehicle and a suspension is produced if the active agent is insoluble. The term “suspension” as used herein is given its ordinary meaning and refers to particles of active agent or aggregates of particles of active agent suspended in the liquid carrier vehicle. When the active agent is present as a suspension the particles of active agent will preferably be in the nano or micron range.

Among the advantages of the present invention is the ability to use a highly-aqueous carrier liquid that is more “bio-friendly” than conventional EHD carriers such as oil-based or solvent-based carriers. The multi-nozzle configuration further enables higher flowrates, while the counter electrodes of the present invention enable design and control over particle size for a given application. A further advantage in many applications is the elimination of slippery or undesired oil or solvent residue from other carrier liquids.

Depending on the biologically-active agent used in the aerosols and methods of the invention, it may be advantageous to include a co-solvent in the aqueous liquid carrier vehicle. The co-solvent may be selected from such groups as alcohols, ethers, alkyl sulfoxides, and propylene oxides. Examples of specific co-solvents include ethanol, 2-ethylhexanol, diacetone alcohol, diisobutyl ketone, isobutanol, isophorone, methyl isobutyl ketone, n-butanol, n-pentanol, n-propanol, and combinations thereof. Ethanol is a particularly preferred solvent because it is soluble in water, is relatively inexpensive, and is safe for the environment, animals, and humans.

The choice of a particular co-solvent or mixture of co-solvents is within the skill of the art and will be made by the skilled artisan taking into account such factors as how an aerosol of the invention will be used, the particular active agent, and if the target surface is a plant, animal, or hard surface. The co-solvent should be soluble in or miscible with water, have a viscosity in the range of 0.1 cPs to 100 cPs, and should not raise the surface tension of the liquid carrier vehicle or aerosol above 60 dynes/cm. The co-solvent will be present in the liquid carrier vehicle of the invention at from about zero weight percent to about 40 weight percent, preferably from about 1 weight percent to about 40 weight percent, and more preferably from about 5 weight percent to about 15 weight percent.

An essential component of the highly-aqueous liquid carrier vehicle of the invention and the aerosols produced therefrom is the surfactant. It important that the surfactant selected be capable of quickly lowering surface tension at the interface between air and liquid as the liquid is exiting the EHD spray nozzle and the electric charge is being applied to the liquid to form the aerosol droplets.

While not being bound by theory, the choice of surfactant or mixtures of surfactants used in the liquid carrier vehicles described herein, it is important to control the surface tension as the aerosol droplet is formed coming from the EHD spray nozzle. It is desirable to keep the surface tension as low as possible at this point in order to produced good aerosolization of the aqueous liquid.

The surfactant, or mixtures of surfactants, used in the aqueous liquid carrier vehicles of the invention should be non-corrosive to the EHD device, should be environmentally safe at the concentrations used, should be non-toxic to humans and animals at the concentrations used, and should have no adverse effect on the activity of the biologically-active agent being delivered in the aerosols of he invention.

Examples of surfactants found to be useful in the aerosols and liquid carrier vehicles of the invention are non-ionics such as alkoxypoly(ethyleneoxy) alcohols such as Rhodasurf® BC 720 (Brenntag, Antwerp, BE), a water-soluble alkoxypoly(ethyleneoxy) ethanol surfactant having an HLB (Hydrophile-Liphophile Balance) of 13.8, alkyl polyglycosides sold under the tradenames Agnique® PG 8107-U (HLB 13.6) and Agnique® PG 9116 (HLB 13.1) (both from Cognis Corp., Cincinnati, Ohio); polyoxyethylene ethers, e.g., polyoxyethylene(10) tridecyl ether (ANAPOE®-C₁₂E₁₀, Anatrace, Maumee, Ohio); alkyl-β-D-glucopyranosides, e.g., hexyl-, heptyl-, octyl-, decyl-, and dodecyl-β-D-glucopyranoside; and alkyl-β-D-maltoglucopyranosides, e.g., hexyl-, octyl-, nonyl-, decyl-, undecyl-, dodecyl-, and tetradecyl-β-D-maltoglucopyranoside (Anatrace).

The choice of a particular surfactant for use in a particular liquid carrier vehicle will be made considering the physical and chemical properties of the active agent to be aerosolized, e.g. whether the active agent is soluble in water or very insoluble, the amount of co-solvent in the liquid carrier vehicle, the nature and amount of any excipient in the liquid carrier vehicle, the desired particle size of the resulting aerosol and the desired spray flow rate. The surfactant will be present in the liquid carrier vehicle of the invention at from about 0.05 weight percent to about 10 weight percent, preferably from about 0.05 weight percent to about 5 weight percent, and more preferably from about 0.1 weight percent to about 2.5 weight percent.

Other optionally-present components in the aerosols and aqueous liquid carrier vehicles of the invention are “biologically-acceptable excipients”. A used herein, the term “biologically-acceptable excipients” include those compounds and additives listed by the FDA as being generally recognized as safe (GRAS) for use in humans (see, 21 CFR § 182). The term also includes those additives that are exempted from the requirement of a tolerance when used in accordance with good agricultural practices. See Federal Insecticide, Fungicide and Rodenticide Act (FIFRA), 7 U.S.C. §136 et seq. (1996) and 40 C.F.R. § 180.1001.

Illustrative of such excipients include but not limited to polyols e.g., propylene glycol, glycerol, polyvinyl alcohol (PVA), and polyethylene glycol (PEG) having an average molecular weight between about 200 and 4000, antioxidants, e.g., Vitamin E, Vitamin E TPGS (alpha-tocopferol polyethylene glycol 1000 succinate), ascorbic acid, anti-microbials, e.g., parabens, pH-adjusting agents, e.g., sodium hydroxide and hydrochloric acid, viscosity-adjusting agents, e.g., polyvinylpyrrolidone, and ionic materials to add charge to the liquid carrier formulation are contemplated for use herein.

The aerosols and aqueous liquid carrier vehicles of the invention may include minor amounts, that is, up to 10 weight percent, preferably from about 0.05 weight percent to about 5 weight percent, and more preferably from about 0.1 weight percent to about 2.5 weight percent of a “biologically-acceptable excipient”. As used herein, the term “biologically-acceptable excipient” refers not only to a single excipient but also to mixtures of two or more excipients; e.g., an aerosol or aqueous liquid carrier vehicle of the invention might contain an antioxidant, a viscosity-adjusting agent, and an ionic material.

While the selection of any particular biologically-acceptable excipient or mixture of excipients is within the skill of the art, the decision regarding whether to add an excipient, and if so which one, will be made taking into account the purpose of the excipient in a specific aqueous liquid carrier vehicle. Any excipient used in the aerosols or liquid carrier vehicles described herein should have no effect or minimal effect on the sprayability of the aqueous liquid containing the biologically-active agent.

The particle size of the aerosol droplets of the invention should be sufficiently large to ensure that the aerosol particles will not be inhaled by an animal or human. The particle size of the aerosol droplets should average from about 60 μm to about 800 μm, preferably from about 80 μm to about 500 μm, and more preferably about 150 μm to about 350 μm in diameter. The average particle size of the droplets is usually referred to as “mass median diameter” (MMD). It is also important that the corresponding geometric standard deviation (GSD) be low, indicating a monodisperse or nearly monodisperse aerosol. A polydisperse aerosol will contain many aerosol particles that are smaller than the target range and many that are larger. Aerosol particles smaller than about 50 μm might be inhaled or “respired” by animals or humans as the aerosol is being applied to the target surface. On the other hand, if the aerosol particles are larger than about 800 μm the aerosol droplets can coalesce and drip off the target surface wasting the biologically-active agent. It is thus highly desirable that the aerosol be as nearly monodisperse as possible.

The highly-aqueous liquid carrier vehicles and compositions prepared according to the invention have a resistivity of from about 0.05 ohm-m to about 100 ohm-m, preferably from about 0.1 ohm-m to about 10 ohm-m, and more preferably from about 0.25 ohm-m to about 5 ohm-m. The highly-aqueous liquid carrier vehicles and compositions prepared according to the invention have a viscosity of from about 0.1 cPs to about 100 cPs, and a surface tension of from about 20 dynes/cm to about 60 dynes/cm.

Unlike the prior art aqueous liquid carrier vehicles, which are generally aerosolized/ sprayed at relatively low flow rates (on the order of μl/sec), the highly-aqueous liquid carrier vehicle of the invention maybe sprayed at commercially-acceptable flow rates. As an example, if a multiple site nozzle having ten sites is used to produce an aerosol according to the invention, and the flowrate at each site is on the order of 0.5 μl/sec/site to 2.0 μl/sec/site, an overall flow rate of 5 μl/sec to 20 μl/sec would result.

The following examples illustrate the method and various compositions and carrier vehicles described herein. The examples were aerosolized using a linear nozzle assembly (4 metallic nozzle ports plus a “dummy” nozzle at each end) with curvilinear-shaped grounded counter electrodes. The nozzles were constructed of stainless steel tubing, had an outside diameter of 0.8 mm, an inside diameter of approximately 0.5 mm and were on 3.5 mm centers. The counter electrodes were constructed of 0.8 mm tubing and, when installed, measured 12.7 mm from top-of-arc to top-of-arc. Each arc was 15.9 mm start-of-bend to end-of-bend. Each arc was orthogonal to the nozzles and was positioned at various points behind, even with, or in front of the tops of the nozzles.

In the examples below, the term “kV” indicates the voltage applied to each spray site of the nozzle of the EHD device to place a charge on the composition. A high voltage source ranging from 0 to +20 kV and 0 to −20 kV was used. The particle size analysis was performed using a Malvern MasterSizer® X particle size analyzer (Malvern Instruments, Inc., Southborough, Mass.). It has been discovered, furthermore, that a charge of negative polarity may work best for spraying highly aqueous formulations, due, it is thought to the bipolar nature of the water molecule.

In general, the formulations of the invention are prepared by adding the components together and mixing to give a liquid solution, an emulsion, or solid in liquid suspension. If the active agent is soluble in water, the active agent is mixed with the aqueous liquid and the co-solvent, surfactant, and excipient (if any) are added to the aqueous solution and the mixture is shaken or stirred to produce a homogenous solution. Where the active agent is substantially insoluble in the aqueous liquid component of the carrier vehicle and is soluble in the co-solvent, the active agent is added to the co-solvent, mixed, and the mixture is added to the aqueous component of the aqueous carrier vehicle. Where the active agent is only slightly soluble in water and/or the co-solvent, it may be advantageous to disperse fine particles of the active agent in the liquid carrier vehicle in order to achieve the desired concentration of the active in the carrier vehicle.

Reagents

| | | |
|---|---|---|
| Ortho Weed-B-Gon ® (Solaris Group, Monsanto Co., | | |
| San Ramon, CA) | | |
| 2,4-D [2,4-dichlorophenoxy acetic acid] | 3.05 | wt % |
| MCPP [2-(4-chloro-2-methylphenoxy) propionic acid] | 10.6 | wt % |
| Dicamba [3,6-dichloro-2-methoxy benzoic acid] | 1.30 | wt % |
| Inerts | 85.05 | wt % |
| C-9 [n-nonyl-β-D-glucopyranoside] | | |
| C-10 [n-decyl-β-D-glucopyranoside] | | |
| Saline [phosphate-buffered saline] | | |
| Emulphogene ® (Sigma-Aldrich, St. Louis, MO) | | |
| [polyoxyethylene-10-tridecyl ether] | | |
| EtOH [ethanol] | | |
| Agnique ® PG 8107-G (Cognis, Cincinnati, OH) | | |
| [C₈-C₁₀ alkyl polyglucoside] | | |
| Agnique ® PG 9116 (Cognis, Cincinnati, OH) | | |
| [C₉-C₁₁ alkyl polyglucoside] | | |
| PBS [phosphate buffer solution] | | |
| phosphate | 10 | mM |
| NaCl | 150 | mM |
| Desonic ® DA-4 (Chemtura, Middlebury, CT) | | |
| [ethoxylated iso-decyl alcohol] | | |

### EXAMPLE 1

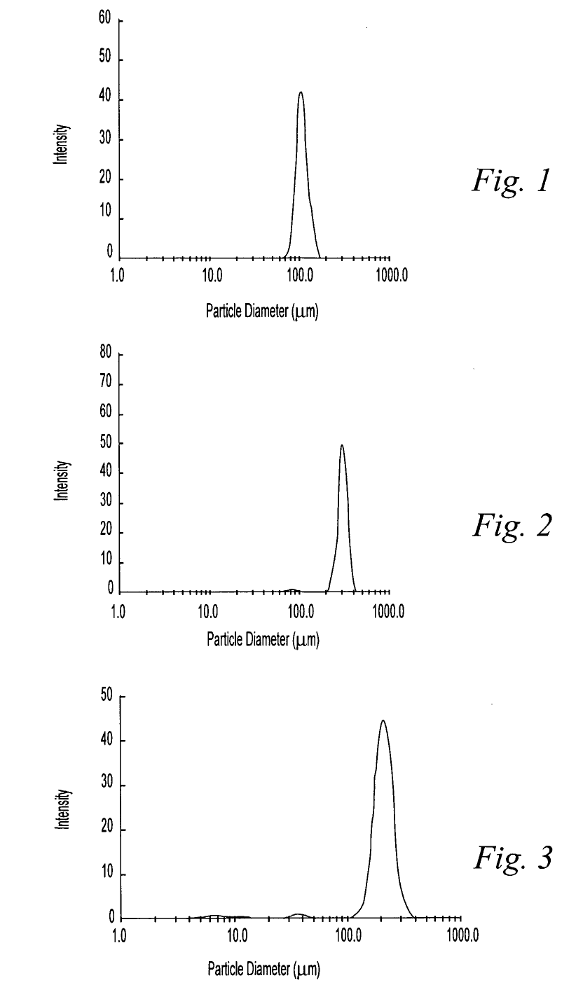

Composition (wt %)

| | Weed-B-Gon ® | 10.90 |
|---|---|---|
| | EtOH | 22.35 |
| | H₂O | 65.75 |
| | C-10 | 1.00 |
| Resistivity (ohm-m) | 1.59 | |
| Surface Tension (dynes/cm) | 36.9 | |
| Viscosity (cPs) | ND | |
| Flowrate (μl/sec/site) | 1.04 | |
| Mass Median Diameter | 107 | |
| (MMD) (μm) | | |
| Geometric Standard | 1.18 | |
| Deviation (GSD) | | |
| Nozzle Configuration | Four-nozzle linear array with an | |
| | additional “dummy” electrode at | |
| | each end and linear parallel ground | |
| | electrodes. | |
| Voltage (kV) | −8.2 | |
| Particle Size Data | FIG. 1 | |

As shown by Ex. 1 and accompanying FIG. 1, no particle sizes less than 60 μm were produced. Satisfactory results were also obtained at flowrates of 0.52 and 0.625 μl/sec/site. MMDs of up to 376 μm with a GSD of 1.29 were observed. Samples with lower concentrations of EtOH (3 weight percent and 6 weight percent) did not spray well and were not analyzed for MMD and GSD.

### EXAMPLE 2

Composition (wt %)

| | Weed-B-Gon ® | 99 |
|---|---|---|
| | C-10 | 1 |
| Resistivity (ohm-m) | 0.485 | |
| Surface Tension (dynes/cm) | 39.3 | |
| Viscosity (cPs) | 1.96 | |
| Flowrate (μl/sec/site) | 1.25 | |
| MMD (μm) | 320 | |
| GSD | 1.22 | |
| Nozzle Configuration | Four-nozzle linear array with an | |
| | additional “dummy” electrode at each end | |
| | and curvilinear ground electrodes. | |
| Voltage (kV) | +9.2 | |
| Particle Size Data | FIG. 2 | |

As shown by Ex. 2 and accompanying FIG. 2, less than two percent of the particles were less than 60 μm. Satisfactory results were also obtained at flowrates of 0.625, 1.375, and 2.75 μl/sec/site.

### EXAMPLE 3

Composition (wt %)

| | Weed-B-Gon ® | 100 |
|---|---|---|
| Resistivity (ohm-m) | 0.46 | |
| Surface Tension (dynes/cm) | 41.9 | |
| Viscosity (cPs) | 1.86 | |
| Flowrate (μl/sec/site) | 1.375 | |
| MMD (μm) | ND | |
| GSD | ND | |
| Nozzle Configuration | Four-nozzle linear array with an | |
| | additional “dummy” electrode at each end | |
| | and curvilinear ground electrodes. | |
| Voltage (kV) | +8.5 and +9.0 | |
| Particle Size Data | ND | |

Although satisfactory sprays were obtained, they were less satisfactory than those observed when a surfactant was added.

### EXAMPLE 4

Composition (wt %)

| | Weed-B-Gon ® | 99 |
|---|---|---|
| | Agnique PG 9116 ® | 1 |
| Resistivity (ohm-m) | 0.49 | |
| Surface Tension (dynes/cm) | 33.8 | |
| Viscosity (cPs) | 1.86 | |
| Flowrate (μl/sec/site) | 1.04 | |
| MMD (μm) | 190 | |
| GSD | 1.37 | |
| Nozzle Configuration | Four-nozzle linear array with an | |
| | additional “dummy” electrode at each end | |
| | and curvilinear ground electrodes. | |
| Voltage (kV) | +8.9 | |
| Particle Size Data | FIG. 3 | |

As shown by Ex. 4 and accompanying FIG. 3, about two percent of the particles were less than 80 μm. Satisfactory results were also obtained at flowrates of 2.1 and 3.125 μl/sec/site.

### EXAMPLE 5

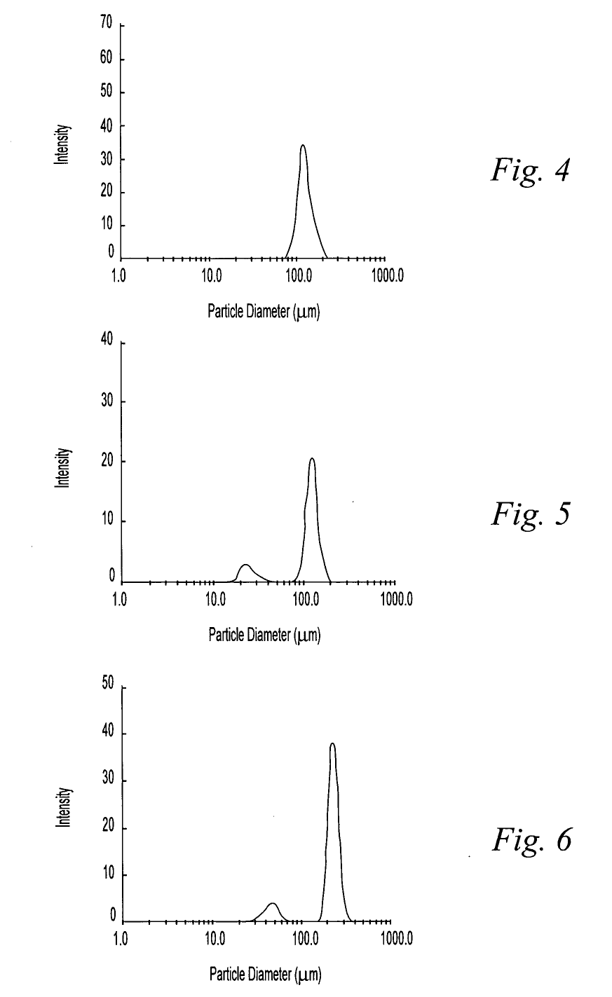

Composition (wt %)

| | PBS | 99 |
|---|---|---|
| | C-10 | 1 |
| Resistivity (ohm-m) | 0.67 | |
| Surface Tension (dynes/cm) | 29.1 | |
| Viscosity (cPs) | 1.00 | |
| Flowrate (μl/sec/site) | 0.83 | |
| MMD (μm) | 156 | |
| GSD | 1.14 | |
| Nozzle Configuration | Four-nozzle linear array with an | |
| | additional “dummy” electrode at each end | |
| | and curvilinear ground electrodes. | |
| Voltage (kV) | +8.6 | |
| Particle Size Data | FIG. 4 | |

As shown by Ex. 5 and accompanying FIG. 4, no particle sizes less than 60 μm were produced. Both linear parallel and curvilinear ground electrodes gave satisfactory sprays. Satisfactory results were also obtained at flowrates of 0.42, 1.04, and 2.08 μl/sec/site.

### EXAMPLE 6

Composition (wt %)

| | PBS | 100 |
|---|---|---|
| Resistivity (ohm-m) | 0.6 | |
| Surface Tension (dynes/cm) | 67.8 | |
| Viscosity (cPs) | 1.16 | |
| Flowrate (μl/sec/site) | ND | |
| MMD (μm) | ND | |
| GSD | ND | |
| Nozzle Configuration | Formulation could not be | |
| | sprayed using any configuration. | |
| Voltage (kV) | NA | |
| Particle Size Data | ND | |

Ex. 6 seems to indicate a surfactant is needed to spray PBS.

### EXAMPLE 7

Composition (wt %)

| | DMA salts of 2,4-D | 3.05 |
|---|---|---|
| | MCPP | 10.6 |
| | Dicamba | 1.30 |
| | The above were dissolved in | |
| | PBS and one percent C-10. | |
| Resistivity (ohm-m) | 0.38 | |
| Surface Tension (dynes/cm) | 37.5 | |
| Viscosity (cPs) | 1.71 | |
| Flowrate (μl/sec/site) | 1.04 | |
| MMD (μm) | ND | |
| GSD | ND | |
| Nozzle Configuration | Four-nozzle linear array with an | |
| | additional “dummy” electrode at each end | |
| | and curvilinear ground electrodes. | |
| Voltage (kV) | +9 | |
| Particle Size Data | ND | |

At flowrates of 0.42 and 1.04 μl/sec/site, a reasonably good spray was produced.

### EXAMPLE 8

Composition (wt %)

| | DMA salts of 2,4-D | 3.05 |
|---|---|---|
| | MCPP | 10.6 |
| | Dicamba | 1.30 |
| | The above were dissolved in | |
| | water with one percent C-10. | |
| Resistivity (ohm-m) | 0.54 | |
| Surface Tension (dynes/cm) | 36.8 | |
| Viscosity (cPs) | 1.66 | |
| Flowrate (μm/sec/site) | 0.42 | |
| MMD (μm) | 187 | |
| GSD | 1.16 | |
| Nozzle Configuration | Four-nozzle linear array with an | |
| | additional “dummy” electrode at each end | |
| | and curvilinear ground electrodes. | |
| Voltage (kV) | +9.8 | |
| Particle Size Data | FIG. 5 | |

As shown by Ex. 8 and accompanying FIG. 5, about 20 percent of the particles were less than 80 μm. Good sprays were also obtained at 1.04 μl/sec/site. At higher flowrates, above 2 μl/sec/site, only jets were obtained with inconsistent sprays.

### EXAMPLE 9

Composition (wt %)

| | Weed-B-Gon ® | 99 |
|---|---|---|
| | Emulphogene ® | 1 |
| Resistivity (ohm-m) | 0.47 | |
| Surface Tension (dynes/cm) | 38.0 | |
| Viscosity (cPs) | 1.68 | |
| Flowrate (μl/sec/site) | 1.04 | |
| MMD (μm) | 179 | |
| GSD | 1.28 | |
| Nozzle Configuration | Four-nozzle linear array with an | |
| | additional “dummy” electrode at each end | |
| | and curvilinear ground electrodes. | |
| Voltage (kV) | +9.7 | |
| Particle Size Data | FIG. 6 | |

As shown by Ex. 9 and accompanying FIG. 6, about 11 percent of the particles were less than 80 μm. Good sprays were also obtained at 0.42 and 1.25 μl/sec/site. At flowrates of 2.5 μl/sec/site and above, arcing and inconsistent sprays resulted.

### EXAMPLE 10

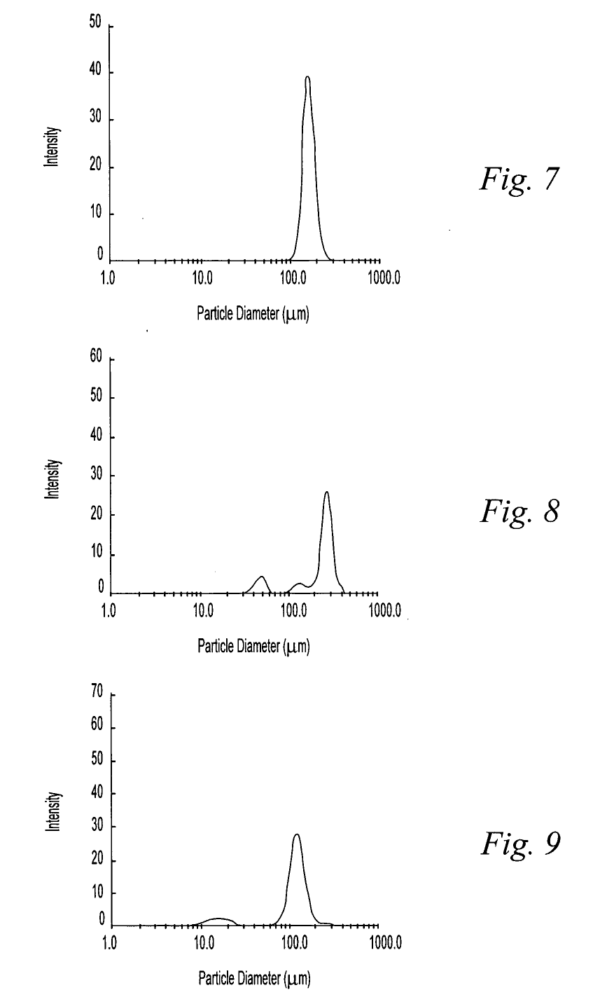

Composition (wt %)

| | Weed-B-Gon ® | 99.9 |
|---|---|---|
| | C-10 | 0.1 |
| Resistivity (ohm-m) | 0.44 | |
| Surface Tension (dynes/cm) | 42.2 | |
| Viscosity (cPs) | 1.65 | |
| Flowrate (μl/sec/site) | 0.83 | |
| MMD (μm) | 175 | |
| GSD | 1.12 | |
| Nozzle Configuration | Four-nozzle linear array with an | |
| | additional “dummy” electrode at each end | |
| | and curvilinear ground electrodes. | |
| Voltage (kV) | +8.7 | |
| Particle Size Data | FIG. 7 | |

As shown by Ex. 10 and accompanying FIG. 7, good sprays are possible with lower concentrations of the C-10 surfactant. Good sprays were also observed at 0.42, 1.25, and 2.5 μl/sec/site, however, higher flowrates (above 3 μl/sec/site) did not yield consistent sprays and only jets were observed.

### EXAMPLE 11

Composition (wt %)

| | Weed-B-Gon ® | 99 |
|---|---|---|
| | Agnique PG 8107 ® | 0.9 |
| | C-10 | 0.1 |
| Resistivity (ohm-m) | 0.45 | |
| Surface Tension (dynes/cm) | 36.6 | |
| Viscosity (cPs) | 1.73 | |
| Flowrate (μl/sec/site) | 0.83 | |
| MMD (μm) | 250 | |
| GSD | 1.61 | |
| Nozzle Configuration | Four-nozzle linear array with an | |
| | additional “dummy” electrode at each end | |
| | and curvilinear ground electrodes. | |
| Voltage (kV) | +9 | |
| Particle Size Data | FIG. 8 | |

Ex. 11 demonstrates the feasibility of using a combination of two different surfactants for spraying aqueous formulations using EHD. Good sprays were also observed at 0.42 and 0.84 μl/sec/site. As shown in FIG. 8, about two percent of the particles were less than 60 μm.

EXAMPLE 12

Composition (wt %)

| | Weed-B-Gon ® | 99 |
|---|---|---|
| | Desonic DA-4 | 1 |
| Resistivity (ohm-m) | 0.46 | |
| Surface Tension (dynes/cm) | 35.5 | |
| Viscosity (cPs) | 1.73 | |
| Flowrate (μl/sec/site) | 1.25 | |
| MMD (μm) | 134 | |
| GSD | 1.41 | |
| Nozzle Configuration | Four-nozzle linear array with an | |
| | additional “dummy” electrode at each end | |
| | and curvilinear ground electrodes. | |
| Voltage (kV) | +9.7 | |
| Particle Size Data | FIG. 9 | |

As shown by Ex. 12 and accompanying FIG. 9, good sprays were observed with this formulation. As shown in FIG. 9, less than 15 percent of the particles were below 60 μm. A flowrate of 0.42 was also successful in producing satisfactory sprays.

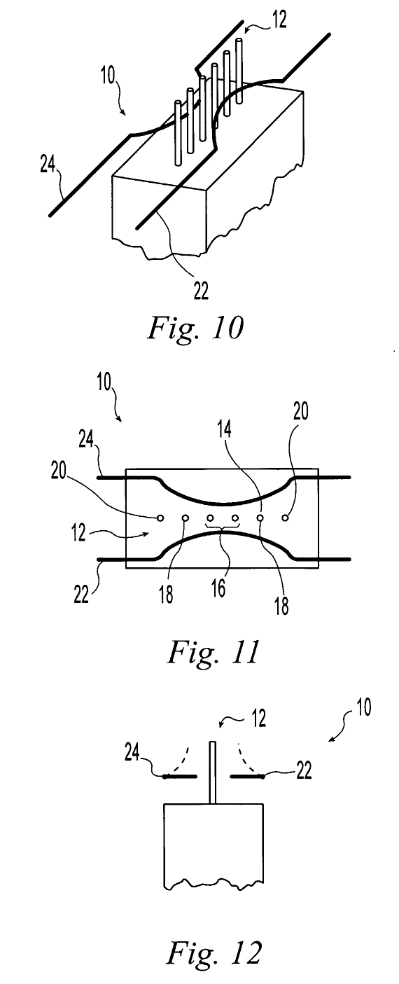

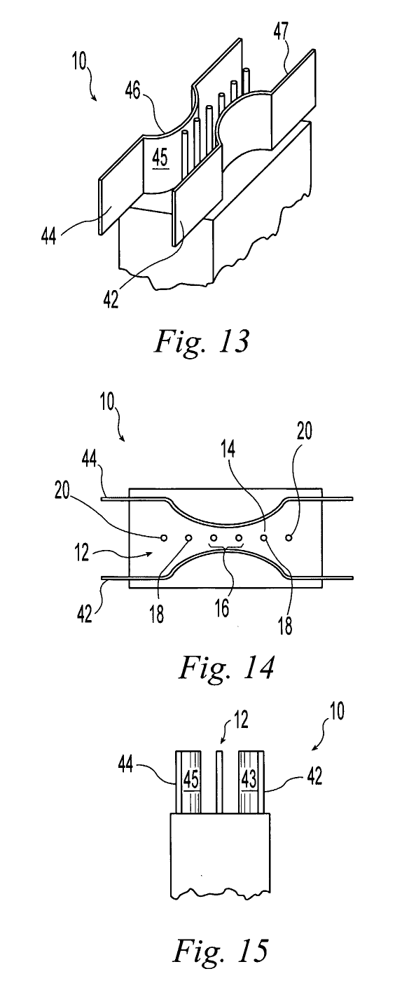

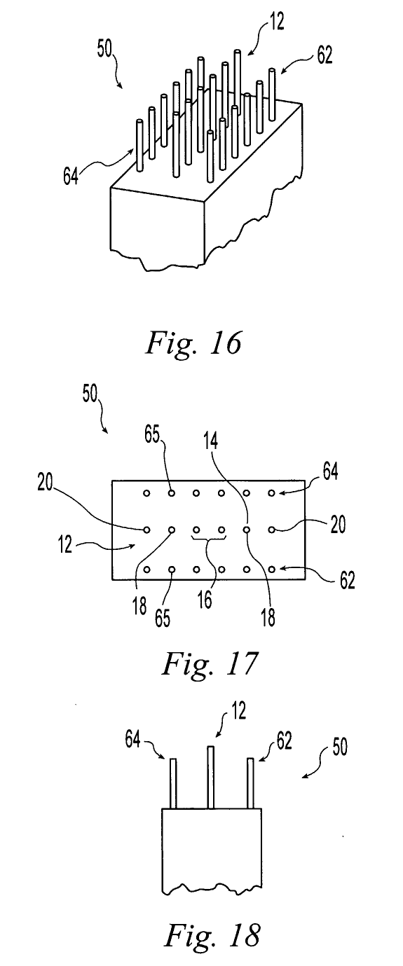

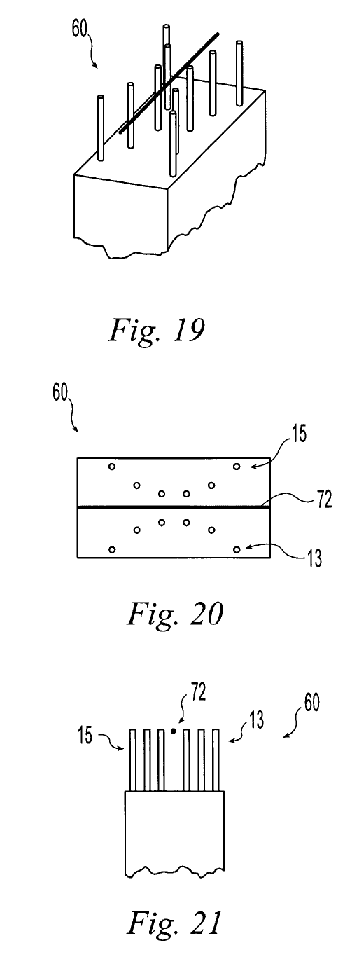

Referring now to FIGS. 10-12, in an EHD nozzle assembly 10 comprising a substantially linear array 12 of individual nozzles 14, it has been found that the nominal central-most spray sites 16 exhibit instability during spraying, especially as the number of nozzles 14 increases. The charge associated with each spray site 14 is affected by the charge associated with nearby spray sites. Thus, the spray sites in the central portion 16 of the array 12 are more affected by nearby spray sites than those in the outer portions 18. As will be appreciated by one skilled in the art, a multi-nozzle array may not be required and a single nozzle 14 may comprise the central-most spray site 16. Additionally, depending upon the charge configuration, as well as other factors, the spray sites denominated “central” 16 and “outer” 18 may change. One or more linear field-equalizing counter electrodes (not shown) have the effect of equalizing the field of the entire array of spray sites. In practice, however, it has been found that the central spray sites 16, being most affected by adjacent nozzles 14, require, relative to the charge of that spray site, a more intense counter charge. This may be effected by exposing the central sites 16 to a more intense field than the outer sites 18. As shown in FIGS. 10-12, this may be accomplished by decreasing the distance between the central spray sites 16 and one or more counter electrodes 22, 24 with a curvilinear counter electrode. As will be appreciated by those skilled in the art, various configurations are potentially effective. The counter electrode 22, 24 may be orthogonal, as shown as solid lines in FIG. 11, or non-orthogonal, as shown as dashed lines in FIG. 11, to the nozzles 12. Further, the field-equalizing effect may be brought about by numerous means. As seen in FIGS. 13-15, a counter electrode 42, 44 may comprise a surface 43, 45 with edges 46, 47 in various configurations relative to the spray sites 14. As shown in FIGS. 16-18, the counter electrode 62, 64 may comprise one or more elements 65 positioned parallel to the nozzles 12. Such a configuration may be further adapted to provide selected charges, or ground, on the individual counter electrode elements 65 as desired. As will be appreciated by one skilled in the art, the counter electrode elements 65 (shown in linear relationship) closest to the central sites 16 may be moved laterally closer (not shown) or may have a different charge to effect the desired countering effect. Finally, FIGS. 19-21 illustrate another embodiment of a nozzle assembly according to the present invention. To properly establish a more equal field at the central nozzles 16, the nozzle arrays 13, 15 may be configured in a curvilinear geometry with the counter electrode 72 positioned proximate.

As well, a curvilinear array of nozzles may be combined with curvilinear counter electrodes or a curvilinear arrangement of counter electrode elements to achieve more uniform spraying in a desired spray pattern.

As will be appreciated by those skilled in the art, a myriad of configurations are possible within the spirit of the invention. By adjusting the field of the spray sites with one or more counter electrodes, spray site uniformity may be improved with commensurate improvement in particle size uniformity at increased fluid flow and increased rates of aerosol delivery.

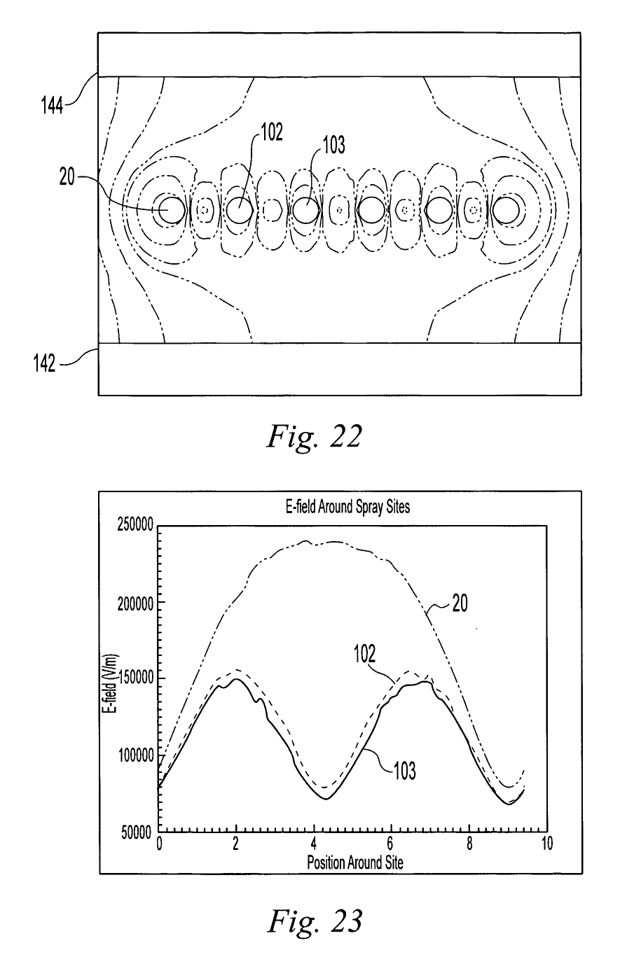

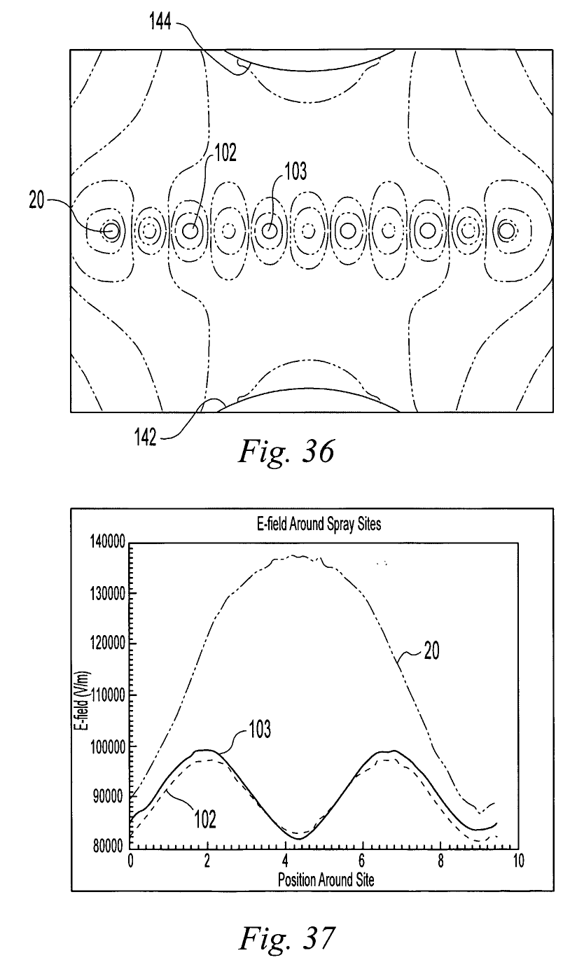

Turning now to FIGS. 22-51, a series of finite element analyses (FEA) were conducted on electric fields associated with a linear array of spray port counter electrodes similar to those discussed above in reference to FIGS. 10-12. As in FIGS. 10-12, one or more “dummy” electrodes 20 are placed at each end of the nozzle array 12 and are not supplied with fluid. The dummy electrodes 20 serve to direct the aerosol generated from the array 12 and eliminate the large electric field variation at the outer sites 18 relative to the central sites 16. The analyses were performed with Maxwell® (Ansoft Corp., Pittsburgh, Pa.), and the models are two-dimensional. The cases discussed below present the parameters associated with the geometry, a plot of the electric field magnitude associated with the geometry, and a graph of the electric field around half of the spray sites 14. Because of symmetry in the models, detail of only half of the sites needs to be presented. Two counter electrodes (nominally 142, 144) are shown above and below the linear array of spray sites 12. Optionally, one counter electrode 142 could be used in the actual nozzle design. The actual nozzle array 12, the spray sites 14, and the counter electrode 142, 144 are in the same plane. These analyses examine the relationships of relative spacings and the shapes of reference electrodes that would produce the most uniform electric field at each spray site 14. Ideally, if the electric field is the same at each port, the formation of the Taylor cone and the aerosol generation will also be uniform. In turn, the droplet or particle size distribution will also be uniform and narrow in its dispersion of sizes.

Table 1, below, summarizes the cases.

TABLE 1

| | | Center-to-Center | Center-to-Edge | Radius | Into Array |
|---|---|---|---|---|---|
| Case | Sites | (mm) | (mm) | (mm) | (mm) |
| 1 | 4 | 5 | 10 | N/A | N/A |
| 2 | 4 | 5 | 20 | N/A | N/A |
| 3 | 4 | 10 | 10 | N/A | N/A |
| 4 | 4 | 10 | 20 | N/A | N/A |
| 5 | 4 | 15 | 10 | N/A | N/A |
| 6 | 4 | 10 | 20 | 80 | N/A |
| 7 | 4 | 10 | 20 | 40 | N/A |
| 8 | 4 | 10 | 20 | 25 | N/A |
| 9 | 10 | 10 | 20 | N/A | N/A |
| 10 | 10 | 10 | 20 | 40 | 20 |
| 11 | 10 | 10 | 30 | 60 | 30 |
| 12 | 10 | 10 | 30 | 30 | 30 |
| 13 | 10 | 10 | 30 | 2.5 | 7.5 |
| 14 | 10 | 10 | 30 | 2.5 | 12.5 |
| 15 | 10 | 10 | 20 | 2.5 | 5 |

In Table 1, the sites noted are active sites. In all cases, the active sites were flanked by one dummy electrode at each end. The Center-to-Edge dimension is the distance from the center of the port (spray site) to the edge of the counter electrode.

In each case, the nozzle ports 14, have an outside diameter (O.D.) of 2 mm which is a median size for many practical ports. Also in each case, because it can be difficult to accurately examine the field directly at the surface of the spray port, an arbitrary circle 0.5 mm beyond the periphery of the port was established to measure and plot the field about each port. Finally, in addition to the sites noted, there is an additional dummy port 20 at each end. (See, e.g., FIG. 22.)

In the detail shown in each case (e.g., FIG. 23), only half of the ports 14, 20 are shown. By adjusting the relationships of relative spacings and the shapes of the counter electrodes, the most uniform field for each port 14 is sought. Of course, the field of the dummy electrode 20 is not relevant. If the field is the same for each port 14 is similar, the formation of the Taylor cone and the aerosol generation will also tend toward uniformity. In turn, the droplet or particle size distribution will also tend toward uniformity and the dispersion will be more narrow.

Seen in FIGS. 22 and 23, Case 1 is the most basic of nozzle configurations. In Case 1, the counter electrodes 142, 144 are twice the port-to-port (center-to-center) spacing from the array 12, linear and parallel to the array 12. Shown in FIG. 23 are the differences in the fields between port 102 and port 103. The reason for a dummy port 101 can be seen in FIG. 20. The differences in the fields of the sites 102, 103 are significant and could cause variations in the aerosol particle size produced.

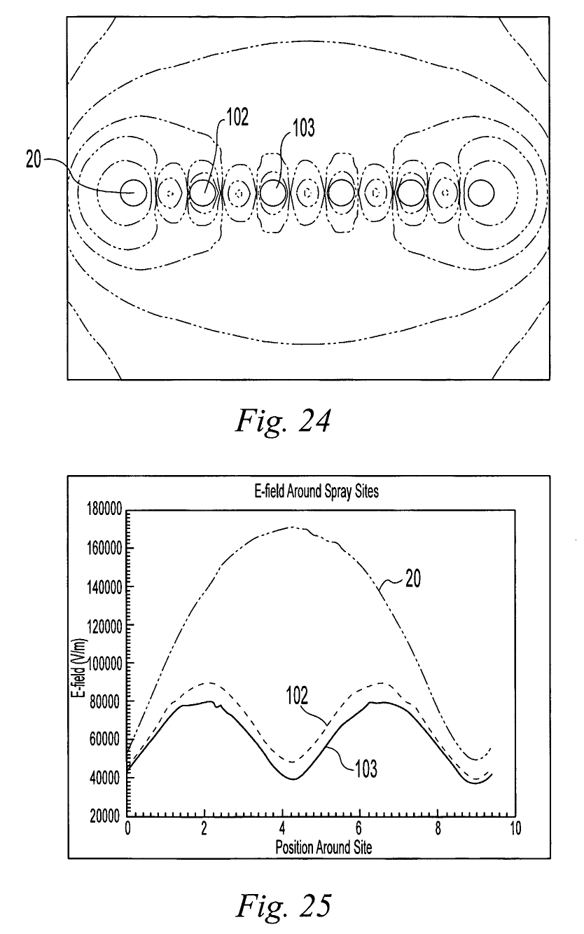

Case 2, shown in FIGS. 24 and 25, illustrates the effect of positioning the counter electrodes (not shown) at four times the port-to-port spacing from the array 12. As shown in FIG. 25, the variations between the sites 102, 103 have increased when compared with Case 1.

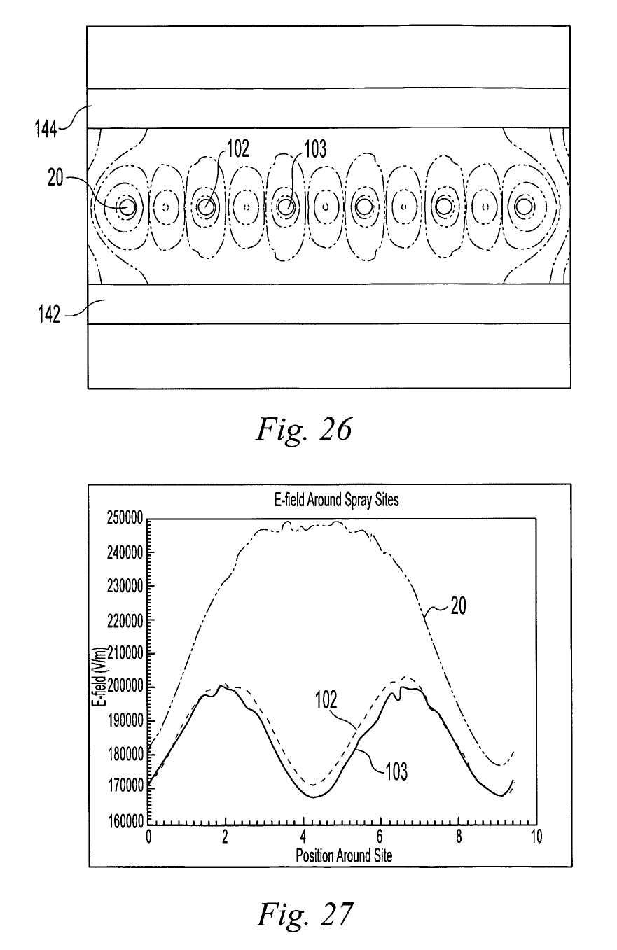

Case 3, shown in FIGS. 26 and 27, illustrates the effect of positioning the counter electrodes 142, 143 at the same distance from the linear array 12 as the port-to-port spacing. As shown in FIG. 27, the variations between the sites 102, 103 have improved, but still larger than preferred.

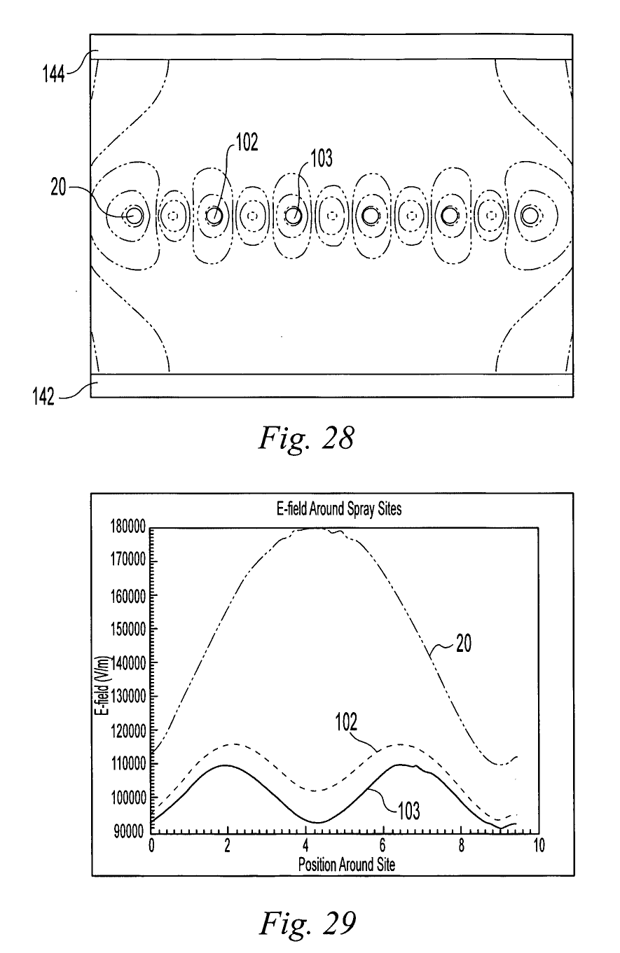

Case 4, shown in FIGS. 28 and 29, illustrates the effect of positioning the counter electrodes 142, 143 at 20 mm, or twice the port-to-port distance of 10 mm. The field disparity has increased from Case 3.

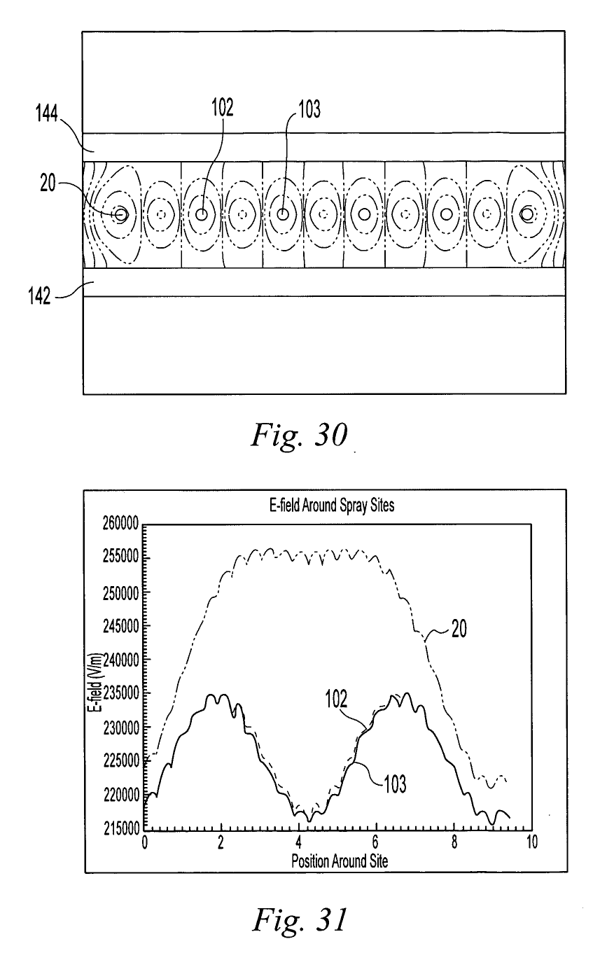

Case 5, shown in FIGS. 30 and 31, illustrates the effect of positioning the counter electrodes 142, 143 at 10 mm, or closer than the adjacent sites 102, 103. This takes relative spacing to the extreme. From FIG. 31, it appears that the sites are beginning to look like independent entities. That is, the field effect from adjacent sites is much less than that of the counter electrodes 142, 143. As a result, as seen in FIG. 31, the fields at the sites 102, 103 appear to be nearly identical.

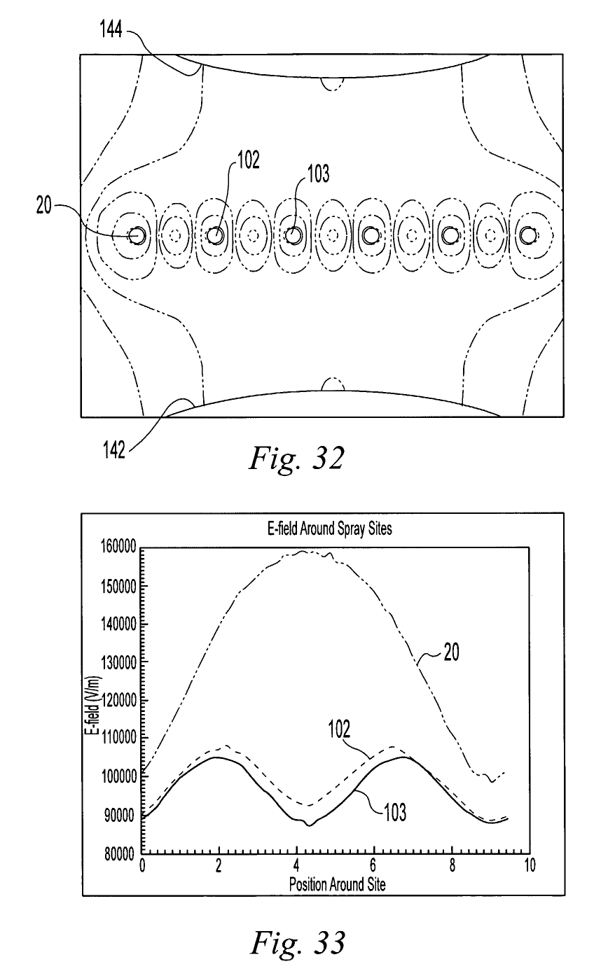

Case 6, shown in FIGS. 32 and 33, is the first model designed to examine the effect of curved counter electrodes 142, 143. The model maintains its symmetry and the minimum counter electrode-site spacing occurs at the midpoint of the linear array 12. For practical purposes, the minimum counter electrode-site spacing was set to twice the site-to-site spacing. This ratio would be adjusted in actual applications.

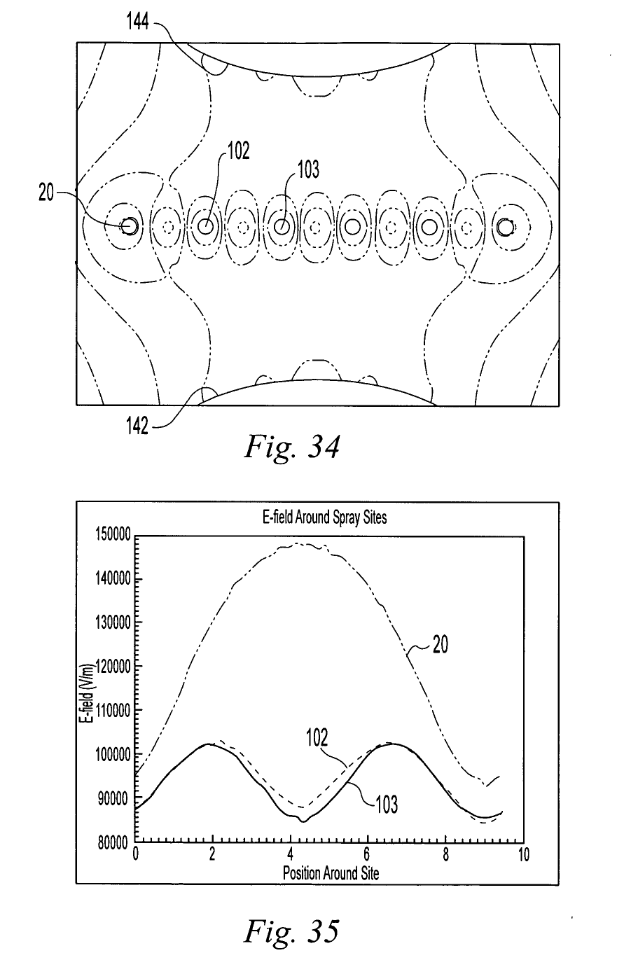

Case 7, shown in FIGS. 34 and 35, illustrates that further reduction in the radius of the counter electrodes 142, 143, further improvement in uniformity results.

Case 8, shown in FIGS. 33 and 34, illustrates the effect of further reduction in the radius of the counter electrodes 142, 143. The disparity in the field increases at a different location around the spray sites 103, 103. (Compare with Case 7.)

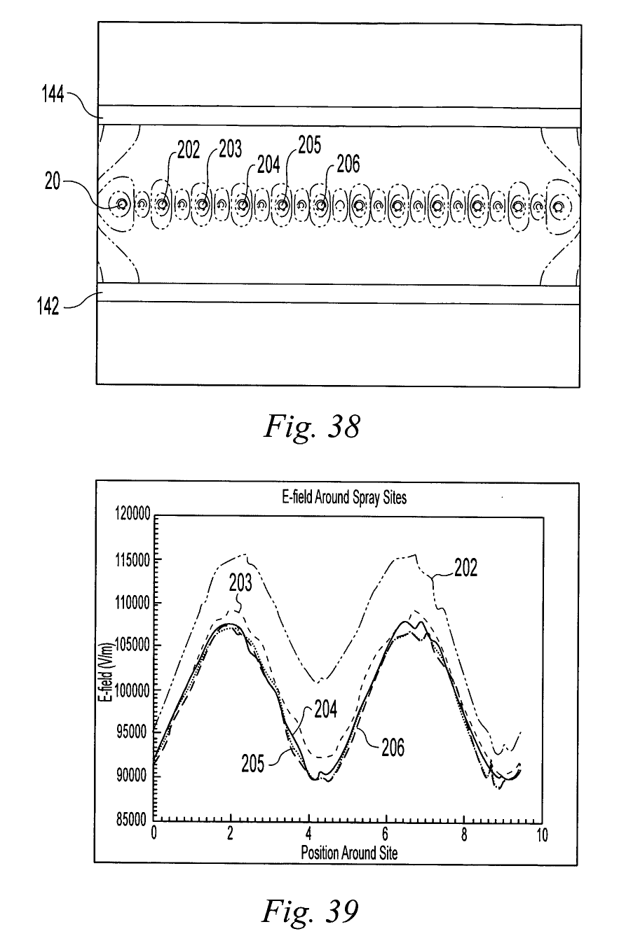

Case 9, shown in FIGS. 38 and 39, is the first model designed to examine a more complex linear array 12. As seen in FIG. 35, there are ten active sites 14 plus two dummy electrodes 20. In FIG. 39, the field magnitude is not studied for the dummy site 20 since it has no liquid and, thus, no Taylor cone. The sites closer to the middle of the array 12, the so-called inner sites 16 experience similar conditions and, therefore, exhibit similar fields. The sites at the outermost positions of the array 12, the so-called outer sites 18 experience more edge effects. Thus, the fields around the outer sites 18 are increasingly disparate from the inner sites 16. Note, again, as will be appreciated by one skilled in the art, that the designation of sites in the array 12 as inner 16 and outer 18 is somewhat arbitrary and is provided as a convenience for analysis and discussion only.

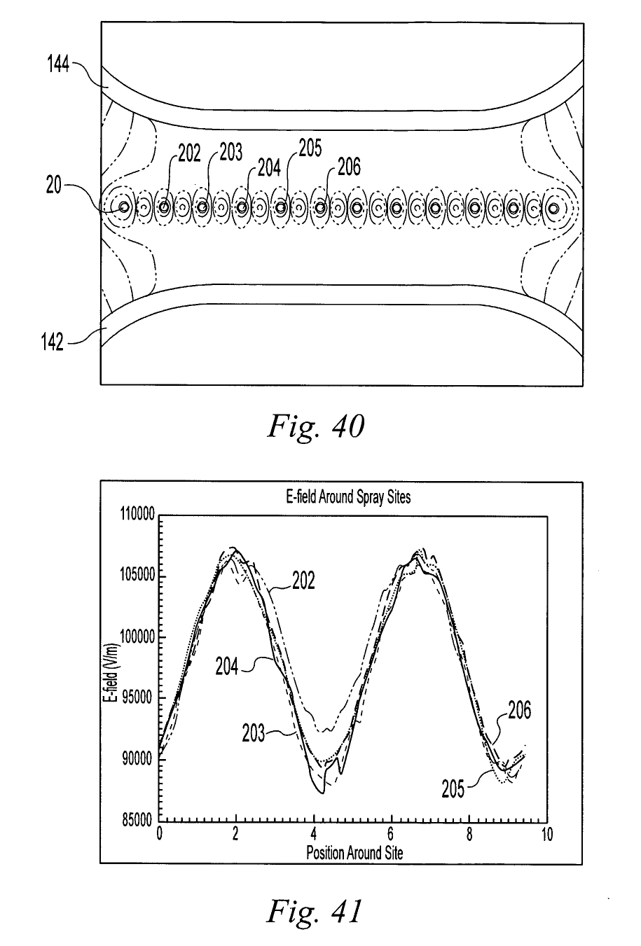

Case 10, shown in FIGS. 40 and 41, illustrates further development in the concept of using counter electrodes 142, 143 to balance the fields around each site 14. Since the inner sites 16 of the array 12 exhibit substantial uniformity, there was no alteration of the counter electrode-spray site geometry. However, based upon Case 9, the intensity of the field around the outer sites 18 have room for improvement. As seen in FIG. 38, there is significant improvement to the uniformity of all sites. Importantly, the counter electrodes 142, 144 begin to curve away from the array 12 at a point “inboard” from the outermost nozzles, especially the active nozzles.

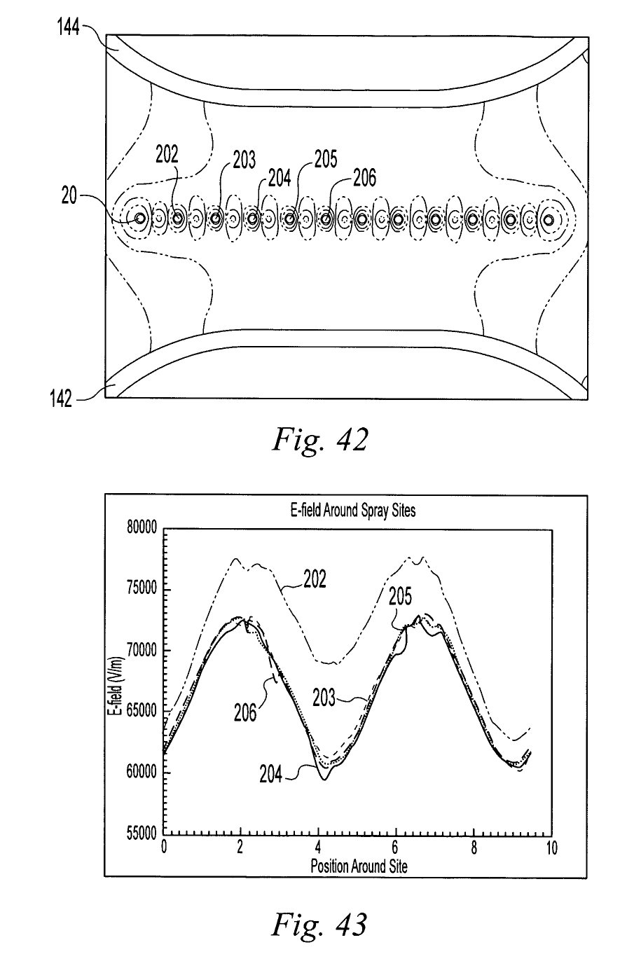

Case 11, shown in FIGS. 42 and 43, illustrates the effect of beginning the radius of curvature of the counter electrodes 142, 143 further inboard from the dummy ports 20. As seen in FIG. 43, the field intensities for all but the most outboard site are very similar. As with Case 10, the counter electrodes 142, 144 begin to curve away from the array 12 at a point “inboard” from the outermost nozzles.

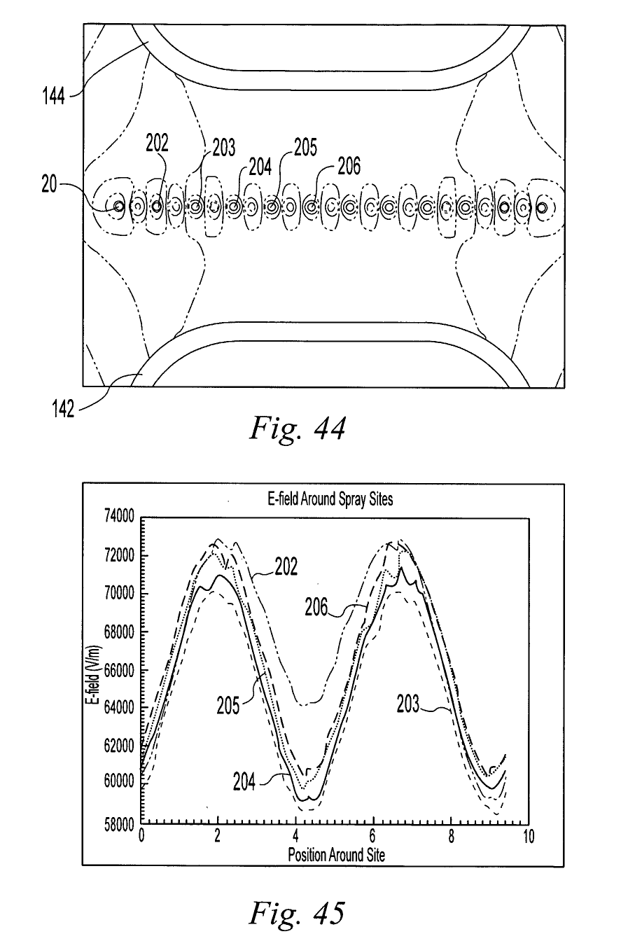

Case 12, shown in FIGS. 44 and 45, illustrates the effect of further increasing the radius of curvature of the counter electrodes 142, 143. While some improvement is made to the outer sites 18, the disparity of the other, inner sites 16, has increased. As with Case 10, the counter electrodes 142, 144 begin to curve away from the array 12 at a point “inboard” from the outermost nozzles.

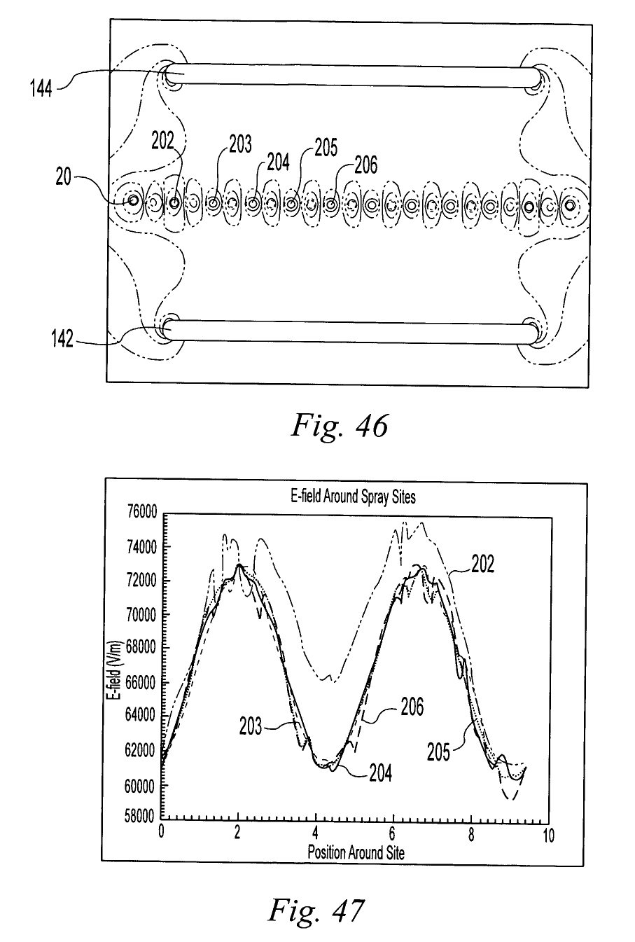

Case 13, shown in FIGS. 46 and 47, illustrates the effect of reducing the curvature of the counter electrode 142, 143 to rounding the edges of the electrode itself. In Case 13, the counter electrodes 142, 144 are defined as flat and having a thickness of 5 mm and ends that are rounded to the thickness diameter of 5 mm. The rounding shown in FIG. 46 helps prevent a high field intensity on the edge of the counter electrode 142, 143. This may also be accomplished, for example, by adding a “bead” to each end or using sheet metal for the counter electrodes 142, 143, and rolling the end over. As with Cases 10-12, the end of the counter electrodes 142, 144 is positioned inboard of the outer nozzles.

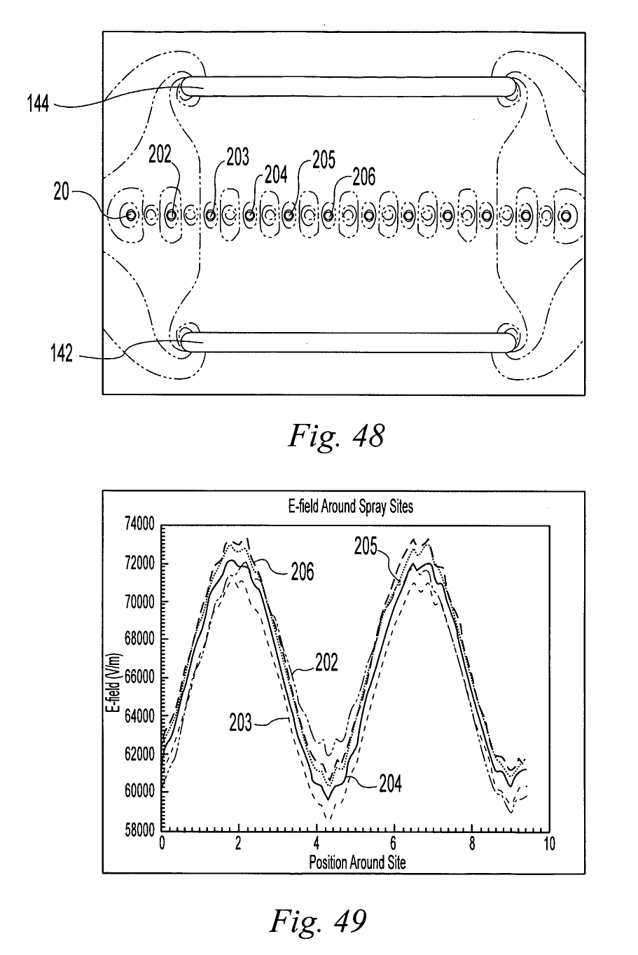

Case 14, shown in FIGS. 48 and 49, illustrates the effect of reducing the length of the counter electrodes 143, 142 relative to the array 12. As seen in FIG. 49, there are diminishing returns to this geometry modification.

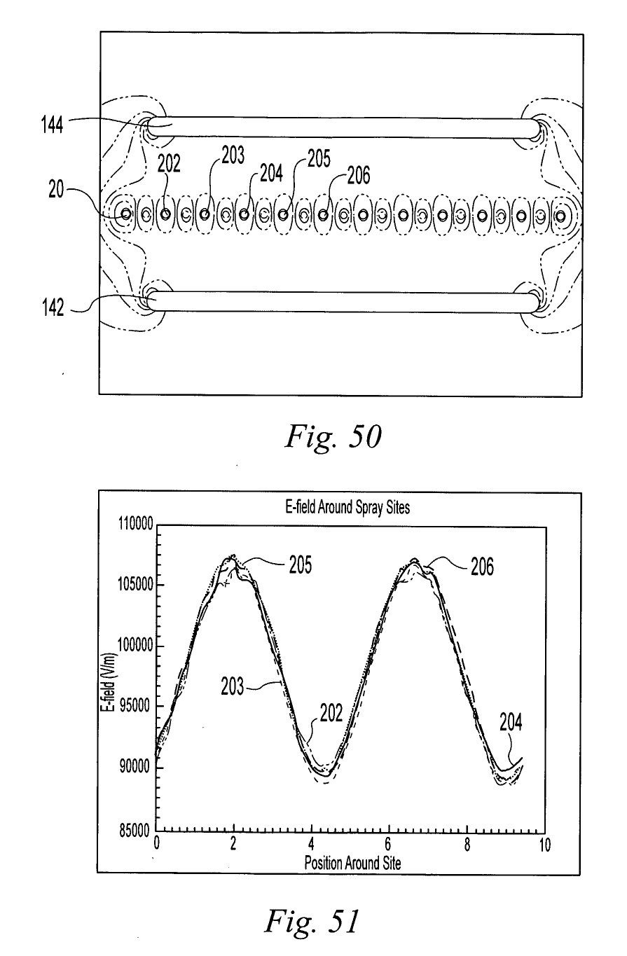

Case 15, shown in FIGS. 50 and 51, illustrate a desirable result; the fields of all spray sites 14 are nearly identical. In relative terms, it was found that the most effective geometry is one where the spacing between the counter electrodes 142, 143 and the array 12 is twice the spacing between the individual sites 14. The ends of the counter electrodes 142, 143 are located midway between the dummy electrode 20 and the first active spray site.

One skilled in the art will readily appreciate that the present invention is well adapted to carry out the objects and obtain the ends and advantages mentioned, as well as those objects, ends, and advantages inherent herein. The present examples, along with the methods, procedures, treatments, specific active agents, and devices described herein, are presently representative of preferred embodiments, are exemplary, and are not intended as limitations on the scope of the invention. Changes therein and other uses will occur to those skilled in the art which are encompassed within the spirit of the invention as defined by the scope of the claims.

We claim:

1. An aqueous liquid carrier vehicle for direct delivery of an aerosol having a particle size of between about 60 μm to about 800 μm, the liquid carrier vehicle comprising:
about 60 wt % to about 100 wt % water;
about 0 wt % to about 40 wt % of a co-solvent;
about 0.05 wt % to about 10 wt % of an acceptable surfactant; and
about 0 wt % to about 10 wt % of an excipient; wherein:
the aerosol is produced using EHD means; and
the liquid carrier vehicle has a resistivity of about 0.05 ohm-m to about 100 ohm-m, a surface tension of about 20 dynes/cm to about 72 dynes/cm, and a viscosity of about 0.1 cPs to about 100 cPs.

2. The liquid carrier vehicle of claim 1, wherein:
the particles have a GSD of less than about 1.65.

3. The liquid carrier vehicle of claim 1, wherein:
the liquid carrier vehicle contains about 70 wt % to about 99 wt % water.

4. The liquid carrier vehicle of claim 3, wherein:
the liquid carrier vehicle contains about 90 wt % to about 95 wt % water.

5. The liquid carrier vehicle of claim 1, wherein:
the liquid carrier vehicle contains about 5 wt % to about 10 wt % of the co-solvent.

6. The liquid carrier vehicle of claim 1, wherein:
the liquid carrier vehicle contains about 99 wt % water and about 1 wt % of the co-solvent.

7. The liquid carrier vehicle of claim 1, wherein:
the co-solvent has a surface tension of about 30 dynes/cm or less.

8. The liquid carrier vehicle of claim 1, wherein:
the co-solvent is selected from the group consisting essentially of: ethanol, 2-ethylhexanol, diacetone alcohol, diisobutyl ketone, isobutanol, isophorone, methyl isobutyl ketone, n-butanol, n-pentanol, n-propanol, polyalcohols, propylene glycol, polyethylene glycol, glycerol, and combinations thereof.

9. The liquid carrier vehicle of claim 8, wherein:
the co-solvent is ethanol.

10. The liquid carrier vehicle of claim 1, wherein:
the liquid carrier vehicle contains about 0.05 wt % to about 5 wt % of the surfactant.

11. The liquid carrier vehicle of claim 10, wherein:
the liquid carrier vehicle contains about 0.1 wt % to about 2.5 wt % of the surfactant.

12. The liquid carrier vehicle of claim 11, wherein:
the liquid carrier vehicle contains about 1 wt % of the surfactant.

13. The liquid carrier vehicle of claim 1, wherein:
the surfactant is selected from the group consisting essentially of: glycosides, polyoxyethylene ethers, alkyl-β-D-glucopyranosides, polyoxyethylene 10 tridecyl ether, ethoxylated iso-decyl alcohol, and alkyl-β-D-maltoglucopyranosides.

14. The liquid carrier vehicle of claim 1, wherein:
the particle size is between about 100 μm to about 500 μm.

15. The liquid carrier vehicle of claim 14, wherein:
the particle size is between about 150 μm to about 200 μm.

16. The liquid carrier vehicle of claim 1, wherein the liquid carrier vehicle comprises:
about 95 wt % to about 100 wt % water;
about 0 wt % to about 5 wt % of the co-solvent;
about 0.1 wt % to about 2.5 wt % of the surfactant; and
about 0.1 wt % to about 2.5 wt % of the excipient; and wherein:
the liquid carrier vehicle has a resistivity of about 0.1 ohm-m to about 100 ohm-m;
a viscosity of about 1 cPs to about 40 cPs; and
a surface tension of about 20 dynes/cm to about 50 dynes/cm.

17. The liquid carrier vehicle of claim 16, wherein:
the liquid carrier vehicle has a resistivity of about 0.25 ohm-m to about 5 ohm-m;
a viscosity of about 1.5 cPs to about 40 cPs; and
a surface tension of about 20 dynes/cm to about 40 dynes/cm.

18. An aerosol having a particle size of between about 60 μm to about 800 μm, and comprising:
a biologically-effective amount of a biologically-active agent dissolved, suspended, or emulsified in the aqueous liquid carrier vehicle of claim 1.

19. The aerosol of claim 18, wherein:
the concentration of the biologically-active agent is about 0.1 wt % to about 30 wt %.

20. The aerosol of claim 19, wherein:
the biologically-active agent is selected from the group consisting essentially of: herbicides, plant growth regulators, insecticides, fungicides, miticides, biocides, antibacterials, antivirials, topical antihistamines, ocular decongestants, and disinfectants.

21. The aerosol of claim 18, wherein:
the liquid carrier vehicle contains about 70 wt % to about 99 wt % water.

22. The aerosol of claim 21, wherein:
the liquid carrier vehicle contains about 85 wt % to about 95 wt % water.

23. The aerosol of claim 21, wherein:
the liquid carrier vehicle contains about 1 wt % to about 30 wt % of the co-solvent.

24. The aerosol of claim 23, wherein:
the liquid carrier vehicle contains about 5 wt % to about 15 wt % of the co-solvent.

25. The aerosol of claim 18, wherein:
the co-solvent is selected form the group consisting of ethanol, 2-ethylhexanol, diacetone alcohol, diisobutyl ketone, isobutanol, isophorone, methyl isobutyl ketone, n-butanol, n-pentanol, n-propanol, and combinations thereof.

26. The aerosol of claim 25, wherein:
the co-solvent is ethanol.

27. The aerosol of claim 18, wherein:
the liquid carrier vehicle contains about 0.05 wt % to about 5 wt % of the surfactant.

28. The aerosol of claim 27, wherein:
the liquid carrier vehicle contains about 0.1 wt % to about 2.5 wt % of the surfactant.

29. The aerosol of claim 28, wherein:
the liquid carrier vehicle contains about 1 wt % of the surfactant.

30. The aerosol of claim 18, wherein:
the surfactant is selected from the group consisting of alkyl polyglycosides, polyoxyethylene ethers. alkyl-β-D-glucopyranosides and alkyl-β-D-maltoglucopyranosides.

31. The aerosol of claim 18, wherein:
the particle size is between about 100 μm to about 500 μm.

32. The aerosol of claim 31, wherein:
the particle size is between about 150 μm to about 250 μm .

33. The aerosol of claim 18, wherein:
the liquid carrier vehicle has a resistivity of about 0.1 ohm-m to about 10 ohm-m;
a viscosity of about 1 cPs to about 50 cPs; and
a surface tension of about 20 dynes/cm to about 50 dynes/cm.

34. The aerosol of claim 33 wherein:
the liquid carrier vehicle has a resistivity of about 0.25 ohm-m to about 5 ohm-m;
a viscosity of about 1.5 cPs to about 40 cPs; and
a surface tension of from abut 20 dynes/cm to about 40 dynes/cm.

35. A method for delivering a biologically-active agent to a target surface in need treatment, comprising the steps of:
a. preparing an aqueous liquid carrier vehicle according to claim 1;
b. dissolving, suspending, or emulsifying a biologically-effective amount of the biologically-active agent in the liquid carrier vehicle;
c. producing an aerosol of the solution or suspension using EHD means, wherein:
the aerosol particle size is about 60 μm to about 800 μm; and
d. applying the aerosol to the target surface.

36. The method of claim 35, wherein:
the concentration of the biologically-active agent in the liquid carrier vehicle is about 0.1 wt % to about 30 wt %.

37. The method of claim 36, wherein:
the biologically-active agent is selected from the group consisting essentially of herbicides, plant growth regulators, insecticides, fungicides, miticides, biocides, antibacterials, antivirials, topical antihistamines, ocular decongestants, and disinfecting agents.

38. The method of claim 35, wherein:
the liquid carrier vehicle contains about 70 wt % to about 99 wt % water.

39. The method of claim 38, wherein:
the liquid carrier vehicle contains about 85 wt % to about 95 wt % water.

40. The method of claim 38, wherein:
the liquid carrier vehicle contains about 1 wt % to about 30 wt % of the co-solvent.

41. The method of claim 40, wherein:
the liquid carrier vehicle contains about 5 wt % to about 15 wt % of the co-solvent.

42. The method of claim 35, wherein:
the co-solvent is selected from the group consisting of ethanol, 2-ethylhexanol, diacetone alcohol, diisobutyl ketone, isobutanol, isophorone, methyl isobutyl ketone, n-butanol, n-pentanol, n-propanol, and combinations thereof.

43. The method of claim 42 wherein the co-solvent is ethanol.

44. The method of claim 35, wherein:
the liquid carrier vehicle contains about 0.05 wt % to about 5 wt % of the surfactant.

45. The method of claim 44, wherein:
the liquid carrier vehicle contains about 0.1 wt % to about 2.5 wt % of the surfactant.

46. The method of claim 45, wherein:
the liquid carrier vehicle contains about 1 wt % of the surfactant.

47. The method of claim 35, wherein:
the surfactant is selected from the group consisting of alkyl polyglycosides, polyoxyethylene ethers, alkyl-β-D-glucopyranosides, and alkyl-β-D-maltoglucopyranosides.

48. The method of claim 35, wherein:
the aerosol particle size is about 80 μm to about 500 μm.

49. The method of claim 35, wherein:
the liquid carrier vehicle has a resistivity of about 2.5 ohm-m to about 5 ohm-m;
a viscosity of about 1.5 cPs to about 40 cPs; and
a surface tension of about 20 dynes/cm to about 40 dynes/cm.

50. A method for delivering a biologically-active agent to a target surface in need treatment, comprising the steps of:
a. providing a biologically-effective amount of the biologically-active agent dissolved, emulsified, or suspended in a liquid carrier vehicle according to claim 1;
b. providing a device according to claim 51;
c. introducing the liquid carrier vehicle containing the agent into the reservoir;
d. producing an aerosol of the solution or suspension using EHD means, wherein:
the aerosol particle size is about 60 μm to about 800 μm; and
f. applying the aerosol to the target surface.

51. A device for producing an aerosol, the device comprising:
a source of a liquid to be aerosolized;
a nozzle array in liquid communication with the source, the nozzle array comprising:
a plurality of nozzles, the plurality of nozzles comprising:
at least one inner nozzle; and
at least one first and at least one second outer nozzle;
an electrical charger, the charger in electrical communication with the liquid or the nozzle array;
at least one counter electrode in charge communication with the liquid or the nozzle array, the counter electrode comprising:
a first end; and
a second end; wherein:
the first end is aligned with, or positioned inboard of, the at least first outer nozzle;
the second end is aligned with, or positioned inboard of, the at least second outer nozzle; wherein:
the liquid or the nozzle array is at a different potential than the counter electrode.

52. The device of claim 51, wherein:
the plurality of nozzles is configured in a substantially linear arrangement.

53. A device for producing an aerosol, the device comprising:
a source of a liquid to be aerosolized;
a nozzle array in liquid communication with the source, the nozzle array comprising:
a plurality of nozzles, the plurality of nozzles comprising:
at least one inner nozzle; and
at least one first and at least one second outer nozzle;
an electrical charger, the charger in electrical communication with the liquid or the nozzle array;
at least one counter electrode, the counter electrode in charge communication with the nozzle array and comprising:
a first end portion;
a second end portion; and
a central portion therebetween; wherein:
the central portion is positioned closer to at least one inner nozzle than the first end portion is positioned to a first outer nozzle, and the central portion is positioned closer to at least one inner nozzle than the second end portion is positioned to a second outer nozzle; and wherein:
the liquid or the nozzle array is at a different potential than the counter electrode.

54. The device of claim 53, wherein:
the plurality of nozzles is configured in a substantially linear arrangement.

55. The device of claim 53, wherein:
the counter electrode comprises a series of discrete electrodes.

56. The device of claim 55, wherein:
the discrete electrodes are aligned in a curvilinear pattern.

57. The device of claim 53, wherein:
at least the first end portion of the counter electrode is substantially curved away from at least the first outer nozzle.

58. The device of claim 53, wherein:
the counter electrode central portion is curved.

59. The device of claim 53, wherein:
the counter electrode central portion is substantially linear.

60. A sprayhead assembly for EHD spraying, the assembly comprising:
a nozzle array, the nozzle array comprising:
a plurality of nozzles, the plurality of nozzles comprising:
at least one inner nozzle; and
at least a first and at least a second outer nozzle;
at least one counter electrode, the counter electrode in charge communication with the nozzle array and comprising:
a first end portion;
a second end portion; and
a central portion therebetween; wherein:
the central portion is positioned closer to at least one inner nozzle than the first end portion is positioned to a first outer nozzle, and the central portion is positioned closer to at least one inner nozzle than the second end portion is positioned to a second outer nozzle; and wherein:
the liquid or the nozzle array is at a different potential than the counter electrode.

61. The sprayhead assembly of claim 60, wherein:
the plurality of nozzles is configured in a substantially linear arrangement.

62. The sprayhead assembly of claim 60, wherein:
the counter electrode comprises a continuous filament.

63. A sprayhead assembly for EHD spraying, the assembly comprising:
a substantially linear counter electrode;
a nozzle array, the nozzle array in charge communication with the counter electrode, and comprising:
a plurality of nozzles, the plurality of nozzles comprising:
at least one central nozzle; and
at least a first and at least a second outer nozzle; wherein:
a central nozzle is positioned closer to the counter electrode than a first outer nozzle is positioned to the counter electrode and a central nozzle is positioned closer to the counter electrode than a second outer nozzle is positioned to the counter electrode.

64. A sprayhead assembly for EHD spraying, the assembly comprising:
a nozzle array, the nozzle array comprising:
a plurality of nozzles, the plurality of nozzles comprising:
at least one central nozzle; and
at least a first and at least a second outer nozzle; and
a plurality of counter electrodes; wherein:
the field intensity of the at least one central nozzle is substantially the same as the field intensity of the at least first and at least second outer nozzle.

65. A sprayhead assembly for EHD spraying, the assembly comprising:
a nozzle array, the nozzle array comprising:
a plurality of nozzles, the plurality of nozzles configured in a substantially linear arrangement and comprising:
at least one central nozzle; and
at least a first and at least a second outer nozzle; and
at least a first, a second, and a third counter electrode; wherein:
the at least one central nozzle is positioned closer to the at least first counter electrode than the at least first outer nozzle is positioned to the second counter and than the at least second outer nozzle is to the third counter electrode.

66. A method of producing an aerosol, the method comprising the steps of:
a. providing a liquid to be aerosolized;
b. providing a nozzle array, the nozzle array comprising:
a plurality of nozzles, the plurality of nozzles configured in a substantially linear arrangement;
c. providing a plurality of counter electrodes;
d. applying a charge to the liquid or to each of the plurality of nozzles;
e. substantially equalizing the field intensity experienced by each of the plurality of nozzles.

67. The method of claim 66, wherein:
the aerosol has a GSD of between about 1.10 and 1.65.

68. The method of claim 66, wherein:
the aerosol is substantially monodisperse.

69. The method of claim 66, wherein:
the liquid is comprises the liquid carrier vehicle of claim 1.

70. A method of substantially equalizing the fields about a plurality of nozzles, the method comprising the steps of:
a. providing a plurality of nozzles;
b. charging at least two of the plurality of nozzles, wherein the charge on the at least two nozzles is unequal;
c. providing at least one counter electrode;
d. applying a charge or ground to the at least one counter electrode, whereby the field on the at least two nozzles becomes substantially equal.
